(19) United States
(12) Patent Application Publication
(10) Pub. No.: US 2025/0122543 A1
(43) Pub. Date: Apr. 17, 2025

(54) CELL-FREE PRODUCTION OF CARMINIC ACID

(71) Applicant: DEBUT BIOTECHNOLOGY, INC., San Diego, CA (US)

(72) Inventors: Neill Kim, San Diego, CA (US); Anthony Agbay, San Diego, CA (US); Jose Amaya, San Diego, CA (US); Jingyi Li, San Diego, CA (US); Nicholas Brideau, San Diego, CA (US)

(21) Appl. No.: 18/756,692

(22) Filed: Jun. 27, 2024

Related U.S. Application Data
(60) Provisional application No. 63/523,725, filed on Jun. 28, 2023.

(51) Int. Cl. C12P 17/02; C12N 9/10; C12N 9/12; C12N 9/90; C12P 7/66; C12P 19/18

(52) U.S. Cl. CPC C12P 17/02; C12N 9/1051; C12N 9/1062; C12N 9/1205; C12N 9/1229; C12N 9/1241; C12N 9/90; C12P 7/66; C12P 19/18; C12Y 204/01013; C12Y 204/0108; C12Y 207/01001; C12Y 207/01002; C12Y 207/04001; C12Y 207/04006; C12Y 207/07009; C12Y 504/02002

(57) ABSTRACT

The invention is related to materials and methods for production of carminic acid from substrates. The invention provides methods and materials for cell-free bioproduction of carminic acid.

### I. FIELD OF INVENTION

The invention is related to materials and methods for production of carminic acid. The invention provides methods and materials for cell-free production of carminic acid.

### II. REFERENCE TO SEQUENCE LISTING

The present application is being filed along with a Sequence Listing in electronic format. The Sequence Listing is provided as a file named DEBU-020-01US.xml, created on Jun. 26, 2024, which is 56 kilobytes in size. The information in the electronic format of the Sequence Listing is incorporated herein by reference in its entirety.

### III. BACKGROUND

Carminic acid is used as an additive in food. Carminic acid is one of the most frequently used dyes in food, medicine, cosmetics and textiles. Carminic acid is added to foods such as ketchup, strawberry milk, and candies. It is also added to cosmetics such as eye shadow, nail polish and lipstick.

Carminic acid is a colorant, which can be extracted from the female insect bodies of Dactylopius coccus costa (alternative name Coccus cacti L.). The insects live on Nopalea coccinellifera, Opuntia fidus indica and other plants of the family Cactaceae cultivated for instance in the desert areas of Mexico, Central and South America and Canary Islands. Depending on the pH the colorant may be a color in a spectrum from orange over red to purple and is generally known as cochineal or cochineal color. Carmine colorant is widely used in foods and beverages.

In relation to current industrial relevant production, carminic acid is harvested by extraction from the insect's dried bodies with water or alcohol. The insects (Dactylopius coccus) are cultured on cacti.

### IV. SUMMARY OF THE INVENTION

The conventional methods of industrial production of carminic acid involves extraction of carminic acid from insect's bodies. In some other convention methods of production of carminic acid, the production of carminic acid may be conducted inside a host organisms and/or cells. As a result, the supply may therefore be relatively expensive and subject to undesirable variations and price fluctuations. The invention provides methods and compositions for cell-free production of carminic acid. The methods provided in the invention related to cell-free production of carminic acid are economic and reliable as compared to other conventional methods of production of carminic acid. In addition, the methods provided in the invention provide higher titer values of the carminic acid from these processes as compared to carminic acid produced from other conventional methods.

In one aspect, the invention provides a cell-free production of carminic acid, wherein the method comprises: providing one or more enzymes in a cell-free medium, wherein the one or more enzymes result in transformation of one or more substrates to carminic acid. In certain embodiments, the substrate that is converted to carminic acid is kermesic acid. In certain embodiments, the enzyme that transforms kermesic acid to carminic acid is C-glucosyltransferase (CGT). In certain embodiments, the reaction medium for C-glucosyltransferase (CGT) mediated enzymatic transformation of kermesic acid to carminic acid further comprises an activated sugar. In certain embodiments, the activated sugar is uridine diphosphate glucose (UDP-glucose). In certain embodiments, UDP-glucose is an essential co-factor for the transformation of kermesic acid to carminic acid. An overview of the conversion of kermesic acid to carminic acid is provided in FIG. 1.

In certain aspects, the activated sugar and/or UDP-glucose is added to the reaction medium. In certain aspects, the activated sugar in the reaction medium for cell-free production of carminic acid is UDP-glucose. In certain embodiments, the UDP-glucose for the reaction medium is generated from sucrose. In certain embodiments, the UDP-glucose is generated in the reaction medium by one or more enzymes. In certain embodiments, the generation of UDP-sugar is mediated by sucrose synthase (SuSy). In certain embodiments, sucrose synthase (SuSy) mediates the conversion of sucrose to UDP-glucose. Thus, in certain embodiments, the reaction medium comprises sucrose, which is subsequently converted to UDP-glucose in course of the reaction. An overview of the process where kermesic acid is converted to carminic acid and UDP-glucose is generated from sucrose is provided in FIG. 2.

In certain beneficial aspects, the invention provides that the UDP-glucose used in the reaction is generated from other substrates in course of the reaction. In certain embodiments, the UDP moiety in UDP-glucose is recycled in course of the process. The recycling of UDP provides economic efficiency of the processes of the invention. Accordingly, in certain aspects, the production of carminic acid from kermesic acid catalyzed by CGT is conducted in conjunction with other methods for UDP-glucose production or recycling of UDP. In certain embodiments, the overview of the synthetic scheme involving the UDP-glucose production and/or recycling is provided in FIG. 3. In certain embodiments, UDP-glucose is generated by the reaction of uridine triphosphate (UTP) with glucose-1-phosphate. In certain preferred embodiments, the reaction of UTP and glucose-1-phosphate is catalyzed by UTP-glucose-1-phosphate uridylyltransferase (UGP). In certain preferred embodiments, the UGP may be galU.

In certain aspects, glucose-1-phosphate is the source for formation of UDP-glucose. Accordingly, in certain embodiments, glucose-1-phosphate may be added to the reaction for generation of carminic acid. In certain embodiments, the glucose-1-phosphate may be supplied to the reaction for preparation of carminic acid.

In certain embodiments, glucose-1-phosphate is generated during the course of the reaction. In certain embodiments, glucose-1-phosphate is generated enzymatically during the course of the reaction. Accordingly, in certain embodiments, glucose is converted to glucose-6-phosphate by a reaction of ATP (adenosine triphosphate) and glucose. In certain embodiments, reaction between glucose and ATP results in generation of glucose-6-phosphate and adenosine diphosphate (ADP). In certain embodiments, conversion of glucose to glucose-6-phosphate is catalyzed by glucokinase (GLK). In certain embodiments, conversion of glucose to glucose-6-phosphate is catalyzed by hexokinase (HK). In certain embodiments, glucose-6-phosphate is converted to glucose-1-phosphate. In certain embodiments, the conversion of glucose-6-phosphate to glucose-1-phsophate is catalyzed by phosphoglucomutase (PGM).

In certain aspects, the invention further provides that the nucleoside triphosphate species used in the reaction are recycled. In certain embodiments, the recycled nucleoside trisphosphates are ATP and UTP. In certain embodiments, UTP is generated by reaction of UDP and ATP. In certain embodiments, the generation of UTP from UDP and ATP is catalyzed by nucleoside diphosphate kinase (NDK). In certain embodiments, ATP may be generated from ADP and phosphate or polyphosphate. In certain embodiments, the conversion of ADP to ATP is catalyzed by polyphosphate kinase (PPK).

In certain beneficial aspects, the invention provides that the processes of the invention are cost effective because of the recycling of chemical intermediates involved in the reaction. In certain embodiments, the invention provides that chemical intermediates like UDP-glucose, UDP, and glucose-1-phosphate are recycled. Indeed, in certain embodiments, the only reagents required in non-catalytic quantities for the production of carminic acid by conversion of kermesic acid are polyphosphate/phosphate and glucose. Because the processes provided in the invention are conducted in a cell free medium and the ingredients (required in non-catalytic quantities) are economic, the processes of the invention are cost effective.

In certain aspects, the one or more enzymes required for the cell-free production of carminic acid are expressed in a host organism. In certain embodiments, the host organism is selected from a group consisting of: bacteria, yeast, and/or mammalian cells. In certain embodiments, the one or more enzymes are introduced in the host organism by integration into genome of the host organism or on a plasmid. In certain embodiments, the plasmid comprises extrachromosomal DNA, which can be expressed by the host organism. In certain embodiments, host organisms expressing the one or more enzymes are cultured until a pre-determined biomass is achieved to produce the requisite quantity of the one or more enzymes. The predetermined biomass is calculated based on the quantity of the one or more enzymes required for the cell-free production of carminic acid. In certain embodiments, the culture comprising host organisms expressing the one or more enzymes are lysed and used as the reaction medium for the methods provided in the invention. In certain other embodiments, once the culture comprising host organisms is lysed, the cell-debris is removed from the lysed matter to prepare the reaction medium for the methods of the invention.

In certain embodiments, the one or more enzymes expressed in the host cells are C-glucosyltransferase (CGT) and/or sucrose synthase (SuSy). In certain embodiments, C-glucosyltransferase (CGT) and/or sucrose synthase (SuSy) are present in the cell-free medium for the cell-free production of carminic acid.

In certain embodiments, the one or more enzymes expressed in host cells are C-glucosyltransferase (CGT), sucrose synthase (SuSy), glucokinase (GLK), hexokinasc (HK), phosphoglucomutase (PGM), polyphosphate kinase (PPK), UTP-glucose-1-phosphate uridylyltransferase (UGP), and/or nucleoside diphosphate kinase (NDK). In certain embodiments, C-glucosyltransferase (CGT), sucrose synthase (SuSy), glucokinase (GLK), hexokinase (HK), phosphoglucomutase (PGM), polyphosphate kinase (PPK), UTP-glucose-1-phosphate uridylyltransferase (UGP), and/or nucleoside diphosphate kinase (NDK) are present in the cell-free medium for the cell-free production of carminic acid.

In certain embodiments of the invention, the reaction mixture comprises purified enzymes for the cell-free production of carminic acid. In certain embodiments, the purified enzymes are C-glucosyltransferase (CGT), sucrose synthase (SuSy), glucokinase (GLK), hexokinase (HK), phosphoglucomutase (PGM), polyphosphate kinase (PPK), UTP-glucose-1-phosphate uridylyltransferase (UGP), and/or nucleoside diphosphate kinase (NDK). In certain embodiments, C-glucosyltransferase (CGT), sucrose synthase (SuSy), glucokinase (GLK), hexokinase (HK), phosphoglucomutase (PGM), polyphosphate kinase (PPK), UTP-glucose-1-phosphate uridylyltransferase (UGP), and/or nucleoside diphosphate kinase (NDK) are not purified. In certain embodiments, the purified enzymes are C-glucosyltransferase (CGT) and/or sucrose synthase (SuSy).

In certain embodiments, the one or more enzymes to produce carminic acid is glucosyltransferase (GT) and/or C-glucosyltransferase (CGT). In certain embodiments, GT and/or CGT used for cell-free production of carminic acid is selected from the enzymes having at least 80% amino acid sequence identity from the enzymes provided in Table 1 below. In certain embodiments, GT and/or CGT used for cell-free production of carminic acid is selected from the enzymes having at least 95% amino acid sequence identity from the enzymes provided in Table 1 below.

TABLE 1

| Accession # | Organism |
|---|---|
| AXF38108 | Bergenia purpurascens |
| AFJ52990 | Linum usitatissimum |
| KAG8368435 | Buddleja alternifolia |
| AFJ52991 | Linum usitatissimum |
| AUI41123 | Rhodiola rosea |
| KAD6795309 | Mikania micrantha |
| XP_006282251 | Capsella rubella |
| BAF18172 | Oryza sativa Japonica Group |
| A0A0B6VIJ5 | Gentiana trifloral |
| GKV43195.1 | Shorea leprosula |
| CAA7037192.1 | Microthlaspi erraticum |
| CAD6239704.1 | Miscanthus lutarioriparius |
| XP_021750757 | Chenopodium quinoa |
| BAG14302 | Gomphrena globose |
| XP_031382117 | Punica granatum |

In certain embodiments, the one or more enzymes involved in production of UDP-glucose, which is an ingredient in the cell-free production of carminic acid is sucrose synthase (SuSy). In certain embodiments, SuSy used for production of UDP-glucose is selected from the enzymes having at least 80% amino acid sequence identity from the enzymes provided in Table 2 below. In certain embodiments, SuSy used for production of UDP-glucose is selected from the enzymes having at least 95% amino acid sequence identity from the enzymes provided in Table 2 below.

TABLE 2

| Accession # | Organism |
|---|---|
| WP_004872341.1 | Acidithiobacillus caldus |
| P30298.2 | Oryza sativa Japonica Group |
| Q820M5.1 | Nitrosomonas europaea ATCC 19718 |
| P13708.2 | Glycine max |
| CAA09297.1 | Nostoc sp. PCC 7119 |
| P49036.1 | Zea mays |
| P49040.3 | Arabidopsis thaliana |
| Q9M111.1 | Arabidopsis thaliana |
| Q8W517 | Ipomoea batatas |
| WP_011056890 | Thermosynechococcus elongatus |

In certain embodiments, the one or more enzymes involved in generation of UDP-glucose and/or recycling of one or more reaction intermediates are provided below. In certain embodiments, the enzymes used for generation of UDP-glucose (by reaction with UTP) and/or recycling of one or more intermediates are selected from the enzymes having at least 95% amino acid sequence identity from the enzymes provided in Table 3 below.

TABLE 3

| Enzyme | Accession No. |
|---|---|
| GLK | AAA27694.1 |
| GLK | WP_331012003.1 |
| PGM | AAC73782.1 |
| PGM | A0A110EI47 |
| PGM | A0A7M2S2U2 |
| UGP | EEW2752841.1 |
| UGP | WP_021648042.1 |
| UGP | WP_028847555.1 |
| NDK | WP_000963837.1 |
| NDK | CAF21035.1 |
| NDK | WP_066795798 |
| PPK2 | WP_010968631 |
| PPK2 | WP_160857702 |

In certain embodiments, the invention provides engineered GT/CGT/SuSy enzymes for the cell-free production of carminic acid. In certain embodiments, the engineered GT/CGT/SuSy enzymes were optimized for cell-free production of carminic acid. In certain embodiments, engineered GT/CGT/SuSy enzymes may include genetic modifications. In certain embodiments, the genetic modifications may be selected from a group consisting of: point mutations, insertions, deletions, and/or any other modifications such that those enzymes result in efficient and optimal cell-free production of carminic acid. In certain embodiments, the GT/CGT/SuSy enzymes used in the cell-free production of carminic acid are any enzymes disclosed in this application.

In certain aspects of the invention, the method of the cell-free production does not require the purification of the one or more enzymes from the lysed host organisms. In certain embodiments, the methods of the invention do not require the purification of one or more enzymes from the lysed biomass comprising host cell expressing the one or more enzymes for the cell-free production of carminic acid. In certain embodiments, the methods of the invention do not require the purification of C-glucosyltransferase (CGT), sucrose synthase (SuSy), glucokinase (GLK), hexokinase (HK), phosphoglucomutase (PGM), polyphosphate kinase (PPK), UTP-glucose-1-phosphate uridylyltransferase (UGP), and/or nucleoside diphosphate kinase (NDK). In certain embodiments, the methods of the invention do not require the purification of C-glucosyltransferase (CGT) and/or sucrose synthase (SuSy) from the lysed cells for cell-free production of carminic acid. This is advantageous because it increases the efficiency of the method and reduces the costs associated with the purification of these enzymes.

In certain embodiments, the cell-free medium may further comprise any other additional ingredients required for the cell-free production of carminic acid. In certain embodiments, the cell-free medium comprises buffer, kermesic acid, an activated sugar, magnesium chloride, cell lysate, sucrose, glucose, glucose-1-phosphate, glucose-6-phosphate, UDP, UTP, ATP, polyphosphate, and/or water. In certain embodiments, the cell-free medium comprises buffer, kermesic acid, an activated sugar, magnesium chloride, cell lysate, sucrose, and/or water. In certain embodiments, the buffer used in the cell-free reaction medium is any buffer suitable for enzymatic conversion of kermesic acid to carminic acid. In certain embodiments, the buffer maintains the pH of about 5 to about 9 in the reaction mixture. In certain embodiments, the buffer maintains the pH of about 6 to about 8 in the reaction mixture. In certain embodiments, the buffer maintains the pH in the range of 6 to 8 in the reaction mixture. In certain embodiments, the buffer is a phosphate buffer. In certain embodiments, the buffer is present at a concentration of about 1 mM to about 200 mM. In certain embodiments, the buffer is present at a concentration of about 5 mM to about 100 mM.

In certain embodiments, the cell-free reaction medium comprises activated sugar at a concentration of about 0.001 mM to about 50 mM. In certain embodiments, the cell-free reaction medium comprises activated sugar at a concentration of about 0.01 mM to about 10 mM. In certain embodiments, the cell-free reaction medium comprises activated sugar at a concentration of about 0.01 mM to about 5 mM. In certain embodiments, the activated sugar is UDP-glucose.

In certain embodiments, the cell-free reaction medium comprises magnesium chloride at a concentration of about 0.5 mM to about 40 mM. In certain embodiments, the cell-free reaction medium comprises magnesium chloride at a concentration of about 1 mM to about 20 mM.

In certain embodiments, the cell-free reaction medium further comprises sucrose. In certain embodiments, the sucrose is converted to UDP-glucose. Thus, the concentration of sucrose in the reaction mixture is determined by the quantity of UDP-glucose needed for the reaction for cell-free production of carminic acid. In certain embodiments, sucrose is present at a concentration of about 10 mM to about 1000 mM in the cell-free reaction medium. In certain embodiments, sucrose is present at a concentration of about 20 mM to about 800 mM in the cell-free reaction medium. In certain embodiments, sucrose is present at a concentration of about 50 mM to about 600 mM in the cell-free reaction medium.

In certain embodiments, the quantity of the one or more enzymes for the cell-free production of carminic acid is dependent on the target quantity of carminic acid to be produced and/or the concentration of other ingredients present in the reaction mixture. In certain embodiments, the one or more enzymes are present in a concentration of from about 1% to about 50% (v/v). In certain embodiments, the one or more enzymes are present in a concentration of from about 2.5% to about 45% (v/v). In certain embodiments, the one or more enzymes are present in a concentration of from about 5% to about 40% (v/v). In certain embodiments, the one or more enzymes are present in a concentration of from about 7.5% to about 30% (v/v).

In certain embodiments, the reaction for production of carminic acid is conducted for a duration till the desired quantity of carminic acid is obtained. In certain embodiments, the reaction for cell-free production of carminic acid is carried out from about 10 minutes to about 48 hours. In certain embodiments, the reaction for cell-free production of carminic acid is carried out from about 10 minutes to about 36 hours. In certain embodiments, the reaction for cell-free production of carminic acid is carried out from about 20 minutes to about 24 hours. In certain embodiments, the reaction for cell-free production of carminic acid is carried out from about 30 minutes to about 20 hours. In certain embodiments, the reaction for cell-free production of carminic acid is carried out from about 1 hour to about 15 hours.

In certain embodiments, the temperature of the reaction mixture for cell-free production of carminic acid is varied to obtain optimal results for the production of carminic acid. In certain embodiments, the temperature of the reaction mixture for cell-free production of carminic acid is from about 15° C. to about 45° C. In certain embodiments, the temperature of the reaction mixture for cell-free production of carminic acid is from about 20° C. to about 40° C. In certain embodiments, the temperature of the reaction mixture for cell-free production of carminic acid is from about 22.5° C. to about 37.5° C. The temperature of the reaction mixture for cell-free production of carminic acid may influence the rate of production of carminic acid. Consequently, the duration of reaction may be adjusted according to the temperature of the reaction mixture to obtain optimal yield of carminic acid in cell-free production of carminic acid.

In certain aspects, the methods provided in the invention may be carried out in any reactor suitable for carrying out the cell-free production of carminic acid. In certain embodiments, the reaction for cell-free production of carminic acid is conducted in a bubble column reactor/bioreactor. In certain embodiments, in the bubble column reactor/bioreactor, the one or more enzymes involved in cell-free production of carminic acid are in a solution. In certain embodiments, the reaction for cell-free production of carminic acid is conducted in a bubble column reactor/bioreactor comprises the lysate from the host organism. In certain embodiments, it is advantageous to use the bubble column reactor/bioreactor for cell-free production of carminic acid when the reaction mixture involves the lysate (or lysate with cellular debris removed) from the host cell organisms in which the one or more enzymes responsible for cell-free production of carminic acid were utilized. In certain embodiments, the reaction for cell-free production of carminic acid is conducted in a packed bed reactor/bioreactor. In certain embodiments, the one or more enzymes are immobilized in the packed bed reactor/bioreactor. The packed bed reactors/bioreactors are preferred for the purified enzymes playing a role in cell-free production of carminic acid. In certain embodiments, the one or more enzymes may be immobilized in a single reactor/bioreactor. In certain other embodiments, the one or more enzymes may be immobilized in different reactors/bioreactors, wherein these reactors/bioreactors are linked sequentially.

The methods provided in the invention are advantageous over other conventional methods of production of carminic acid. In certain embodiments, the methods of the invention provide cell-free production of carminic acid. Because the methods of the invention are conducted in cell-free medium, they provide significant economic efficiency by reducing the cost of production of carminic acid in other conventional methods. In certain embodiments, because the reaction for production of carminic acid is conducted from the lysates from the host organisms expressing the one or more enzymes involved in the reaction, the methods of the invention are cost-efficient. In particular, in certain embodiments, the methods of the invention do not involve the purification of the one or more enzymes. Because purification of individual enzymes is not required in the methods of the invention, provides further economic efficiency by reducing the cost that would have been otherwise required in purifying individual enzymes.

Advantageously, the cell-free production of carminic acid provided herein provides a significantly higher titer value for carminic acid as compared to the conventional methods. The higher titer values of carminic acid provide additional cost advantages for production of carminic acid because the higher titer carminic acid would provide efficiency in purifying and/or concentrating carminic acid from the reaction mixture. In certain embodiments, the methods of the invention provide carminic acid titer value of from at least 5-fold higher than the conventional methods. In certain embodiments, the methods of the invention provide carminic acid titer value of from at least 10-fold higher than the conventional methods. In certain embodiments, the methods of the invention provide carminic acid titer value of from at least 50-fold higher than the conventional methods. In certain embodiments, the methods of the invention provide carminic acid titer value of from at least 100-fold higher than the conventional methods. In certain embodiments, the methods of the invention provide carminic acid titer value of from at least 500-fold higher than the conventional methods. In certain embodiments, the methods of the invention provide carminic acid titer value of from at least 1000-fold higher than the conventional methods. In certain embodiments, the methods of the invention provide carminic acid titer value of from at least 5000-fold higher than the conventional methods.

In certain aspects, the invention provides compositions for cell-free production of carminic acid. The compositions of the invention are utilized for cell-free production of carminic acid in accordance with the methods described above.

In certain embodiments, the compositions of the invention comprise: one or more enzymes in a cell-free medium, wherein the one or more enzymes result in transformation of a substrate to carminic acid. In certain embodiments, the substrate converted to carminic acid is kermesic acid. In certain embodiments, the one or more enzyme is C-glucosyltransferase (CGT). In certain embodiments, the compositions further comprise a cell-free medium. In certain embodiments, the cell-free medium comprises an activated sugar. In certain embodiments, the activated sugar is UDP-glucose. In certain embodiments, the UDP-glucose is added to the cell-free medium.

In certain aspects of the invention, the UDP-glucose in the composition is synthesized in the cell-free medium by the one or more enzymes. In certain embodiments, the UDP-glucose is synthesized from sucrose. In certain embodiments, the one or more enzymes is sucrose synthase (SuSy) that synthesizes UDP-glucose from sucrose. In certain embodiments, the cell-free medium is a cell lysate. In certain embodiments, the cell lysate is a cell lysate from cells from a host organism expressing the one or more enzymes. In certain embodiments, the host organism is selected from a group consisting of: bacteria, yeast, and/or mammalian cells. In certain embodiments, the one or more enzymes are introduced in the host organism by integration into genome of the host organism or on a plasmid. In certain embodiments, the host organisms expressing the one or more enzymes are cultured until a pre-determined biomass is achieved to produce the requisite quantity of the one or more enzymes. In certain embodiments, the composition further comprises cell lysate generated by lysis of cells and subsequent removal of cell debris, for use in the cell-free medium for cell-free production of carminic acid.

In certain embodiments, the CGT, SuSy, GLK, HK, PGM, PPK, UGP, and/or NDK enzymes are present in the cell-free medium for cell-free production of carminic acid. In certain embodiments, the compositions of the invention provide CGT, SuSy, GLK, HK, PGM, PPK, UGP, and/or NDK that have not been purified or separated.

In certain embodiments, the CGT and SuSy enzymes are present in the cell-free medium for cell-free production of carminic acid. In certain embodiments, the compositions of the invention provide CGT and SuSy that have not been purified or separated.

In certain embodiments, the cell-free medium may further comprise any other additional ingredients required for the cell-free production of carminic acid. For example, in certain embodiments, the cell-free medium comprises buffer, kermesic acid, an activated sugar, magnesium chloride, cell lysate, sucrose, and/or water. In certain embodiments, the buffer used in the cell-free reaction medium is any buffer suitable for enzymatic conversion of kermesic acid to carminic acid. In certain embodiments, the buffer maintains the pH of about 5 to about 9 in the reaction mixture. In certain embodiments, the buffer maintains the pH of about 6 to about 8 in the reaction mixture. In certain embodiments, the buffer maintains the pH in the range of 6 to 8 in the reaction mixture. In certain embodiments, the buffer is a phosphate buffer. In certain embodiments, the buffer is present at a concentration of about 1 mM to about 200 mM. In certain embodiments, the buffer is present at a concentration of about 5 mM to about 100 mM.

In certain embodiments, the cell-free reaction medium comprises activated sugar at a concentration of about 0.001 mM to about 50 mM. In certain embodiments, the cell-free reaction medium comprises activated sugar at a concentration of about 0.01 mM to about 5 mM. In certain embodiments, the activated sugar is UDP-glucose.

In certain embodiments, the cell-free reaction medium comprises magnesium chloride at a concentration of about 0.5 mM to about 40 mM. In certain embodiments, the cell-free reaction medium comprises magnesium chloride at a concentration of about 1 mM to about 20 mM.

In certain embodiments, the cell-free reaction medium further comprises sucrose. In certain embodiments, the sucrose is converted to UDP-glucose. Thus, the concentration of sucrose in the reaction mixture is determined by the quantity of UDP-glucose needed for the reaction for cell-free production of carminic acid. In certain embodiments, sucrose is present at a concentration of about 10 mM to about 1000 mM in the cell-free reaction medium. In certain embodiments, sucrose is present at a concentration of about 20 mM to about 800 mM in the cell-free reaction medium. In certain embodiments, sucrose is present at a concentration of about 50 mM to about 600 mM in the cell-free reaction medium.

In certain embodiments, the quantity of the one or more enzymes for the cell-free production of carminic acid is dependent on the target quantity of carminic acid to be produced and/or the concentration of other ingredients present in the reaction mixture. In certain embodiments, the one or more enzymes are present in a concentration of from about 1% to about 50% (v/v). In certain embodiments, the one or more enzymes are present in a concentration of from about 2.5% to about 45% (v/v). In certain embodiments, the one or more enzymes are present in a concentration of from about 5% to about 40% (v/v). In certain embodiments, the one or more enzymes are present in a concentration of from about 7.5% to about 30% (v/v).

In certain aspects of the invention, the compositions of the invention are in a bubble column reactor, wherein the one or more enzymes are in a solution. In certain embodiments, the compositions of the invention are in a packed bed reactor, wherein the one or more enzymes are immobilized.

### V. BRIEF DESCRIPTION OF FIGURES

FIG. 1 provides a schematic of conversion of kermesic acid to carminic acid.

FIG. 2 provides a schematic of the reaction scheme showing the conversion of kermesic acid to carminic acid, and the conversion of sucrose to UDP-glucose.

FIG. 3 provides a schematic of the reaction scheme showing the conversion of kermesic acid to carminic acid, wherein the UDP is recycled.

FIG. 4 provides HPLC chromatograms demonstrating the conversion of kermesic acid to carminic acid in a cell-free medium upon addition of CGT and UDP-glucose in the reaction mixture.

### VI. DETAILED DESCRIPTION

The present application provides compositions and methods for production of carminic acid in a cell-free medium, wherein one or more enzymes in a cell-free medium, wherein the one or more enzymes result in transformation of an organic material to carminic acid. The one or more enzymes may be engineered. The engineered enzyme may be non-naturally occurring.

The term “non-naturally occurring”, when used in reference to an enzyme is intended to mean that nucleic acids or polypeptides include at least one genetic alteration not normally found in a naturally occurring polypeptide or nucleic acid sequence. Naturally occurring nucleic acids, and polypeptides can be referred to as “wild-type” or “original”. A host cell, organism, or microorganism that includes at least one genetic modification generated by human intervention can also be referred to as “non-naturally occurring”, “engineered”, “genetically engineered,” or “recombinant”.

As used herein, the singular forms “a,” “an,” and “the” are intended to include the plural forms as well, unless the context clearly indicates otherwise. Furthermore, to the extent that the terms “including,” “includes,” “having,” “has,” “with,” or variants thereof are used in either the detailed description and/or the claims, such terms are intended to be inclusive in a manner similar to the term “comprising.”

As used herein, “reaction solution” may refer to all components necessary for enzyme-based chemical transformation. This is typically, but not limited to, buffering agent, salts, cofactor, and substrate (starting material).

As used herein, “reaction mixture” may refer to all components from the “reaction solution” plus the enzyme(s) and/or products from the reaction. In some embodiments, the “reaction mixture” may refer to just the reaction solution without any enzymes or reaction products. In some embodiments, “reaction solution” and “reaction mixture” may be used interchangeably.

As used herein, “buffering agents” may refer to chemicals added to water-based solutions that resist changes in pH by the action of acid-base conjugate components.

As used herein, “cofactors” may refer to a non-protein chemical compound that may bind to a protein and assist with a biological chemical reaction. Non-limiting examples of cofactors may include but are not limited to NADPH and NADH.

In the cell-free systems described herein, the critical components of the cell, namely cofactors and enzymes, are used in a chemical reaction without cellular components that can directly or indirectly inhibit the desired biochemical reaction. The same enzymes found in plants and other organisms may be created in vivo (typically through protein overexpression in hosts such as bacteria), isolated via chromatography and/or any other methods, and then added into a bioreactor with a substrate (starting material). The enzymes may also be used directly from plants without any isolation. The enzymes transform the substrate in the same way that occurs in the original organism without the organism's complexity. Additionally, the biochemical reaction may be enhanced by the addition of co-solvents, detergents, or both, which would not be tolerated by, or simply would not work in a whole cell-based manufacturing method. In this way, natural products can be created without the plant, cell, or chemical synthesis.

### Carminic Acid:

Carminic acid is used in edible food colorants. Carminic acid is an anthraquinone compound containing a glucose molecule attached via a glycosidic bond. Unsaturated rings in the anthraquinone portion strongly absorb light and are responsible for its intense purple color. Depending on the pH the colorant may be a color in a spectrum from orange over red to purple and is generally known as cochineal or cochineal color. Carmine is the most used form of carminic acid. Carminic acid and derivatives are found in a large range of food such as ice cream, confectionery, culinary, desserts, beverages, dairy and others. Carmine is also widely used in cosmetics. Carmine is a highly stable pigment that can reasonably withstand heat processing and adverse storage conditions. Carmine, the most used form of carminic acid, is obtained by adding aluminum sulphate in basic conditions to the cochineal extract. This produces a complexation of carminic acid to the metal that cause a bathochromic shift that after sol-gel polymerization gives an insoluble red lake formulation.

The widespread use of carminic acid derivatives in the food and cosmetic industries may be explained in part by the fact that they are one of a few rare exceptions of naturally derived colors exhibiting comparable stability to artificial dyes. Indeed, processing conditions such as heat or storage under light and oxygen have a minor effect on degradation.

The chemical structure of carminic acid is provided below.

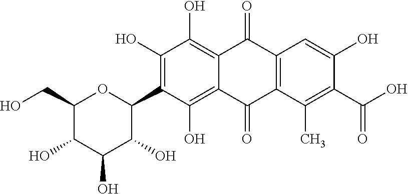 [SMILES: Cc1c(C(=O)O)c(O)cc2c1C(=O)c1c(O)c([C@@H]3O[C@H](CO)[C@@H](O)[C@H](O)[C@H]3O)c(O)c(O)c1C2=O]

### Synthesis of Carminic Acid:

Carminic acid is harvested by extraction from the said insect's dried bodies with water or alcohol. During the aqueous based extraction of carminic acid from the insect, an amount of insect protein is also released from the insect and will be contained in the color extract. The level of insect protein is typically less than 0.5%. The aqueous based extract of cochineal is primarily containing carminic acid plus some cochineal protein and other minor extractable substances from the insect. Hereinafter this extract is referred to as cochineal extract solution.

Harvesting and processing of the insects is not an easy task as it determines the final quality of the color. South American countries such as Peru have ideal climatic conditions to grow cactus plants that are the vital food source for these insects, and thus it is here where carmine is industrially most sourced. Crude orange extracts of carminic acid are thus purified prior to being put into color formulation production. In order to obtain 1 kg of extract for colorants, about 100,000 insects need to be processed.

Due to these poor yields and the fact that until a few years ago artificial colors were not an issue for the industry, local economy was more in favor of a profitable business such as vegetables farming (peppers, asparagus, etc.). Nevertheless, due to the recent trend for natural colors in the food industry, the consumption of carmine increased greatly from 2004 to 2010 and shortage provoked high boost in price never before observed. Since it is derived from animal sources, carmine is often at the center of debate with respect to religious discussions where Halal and Kosher preferences both prohibit the use of this color. Thus, alternative methods of synthesis of carminic acid are required.

WO 2006/056585 describes a process in which the aqueous based extraction of carminic acid from the insect, an amount of insect protein is also released from the insect and will be contained in the color extract and it has been reported that the cochineal insect proteins could create some allergy related problems. WO2006/056585 describes a special process to reduce the amount of insect protein from the insect extract solution is described—however, the final produced color composition/product of WO2006/056585 will still comprise some amounts Dactylopius coccus costa insect proteins.

WO 2022/013659 provides a method for the production of carminic acid from insects of the genus Dactylopius coccus. The method comprising: extracting hemocytes from hemolymph of insects, recover the purified hemocytes; culturing the purified hemocytes with culture medium for cultivation of insect cell lines; stimulate and activate insect cell lines; and propagate insect cell lines by producing carminic acid. However, these methods involve the unpredictability and complication of considering bioproduction in the cell lines.

The invention provides methods and compositions for cell-free production of carminic acid. Because the methods of the invention involve cell-free production of carminic acid, the methods provided in the invention related to cell-free production of carminic acid are economic and reliable as compared to other conventional methods of production of carminic acid. In addition, the methods provided in the invention provide higher titer values of the carminic acid from these processes as compared to carminic acid produced from other conventional methods. The methods of the invention are also advantageous because the cell-free production of carminic acid reduces the risk of degradation of carminic acid after production. In addition, the cell-free synthesis method allows for higher concentration of carminic acid being produced without killing the cell. Moreover, cell-free production provides case of purification of carminic acid after the reaction is complete. In certain aspects, the cell-free production of carminic acid allows for the recycling of enzymes used in the reactions for preparation of carminic acid. Thus, the same batch of enzyme may be used for multiple batches of carminic acid being produced. In certain embodiments, the enzymes may be purified by downstream processing by techniques such as filtration and centrifugation.

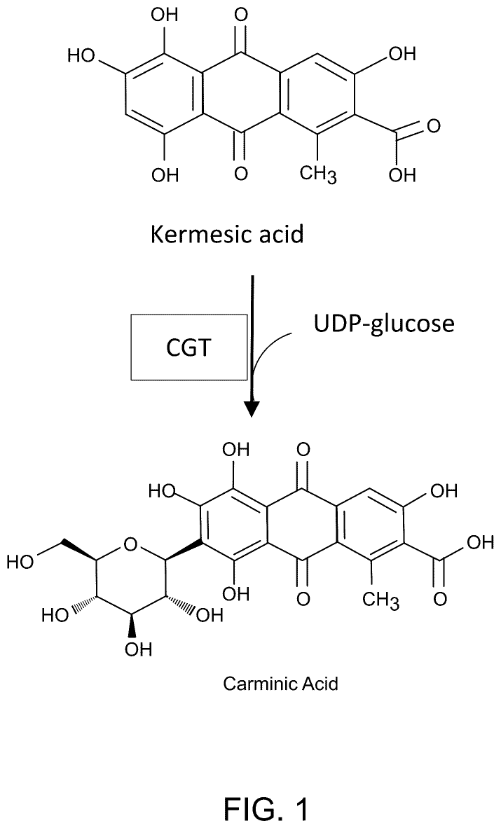

In one aspect, the invention provides a cell-free production of carminic acid, wherein the method comprises: providing one or more enzymes in a cell-free medium, wherein the one or more enzymes result in transformation of a substrate to carminic acid. In certain embodiments, the substrate that is converted to carminic acid is kermesic acid. In certain embodiments, the enzyme that transforms kermesic acid to carminic acid is C-glucosyltransferase (CGT). The CGT mediated conversion of kermesic acid to carminic acid is provided in FIG. 1. In certain embodiments, the reaction medium for C-glucosyltransferase (CGT) mediated enzymatic transformation of kermesic acid to carminic acid further comprises an activated sugar. In certain embodiments, the activated sugar is UDP-glucose. In certain embodiments, UDP-glucose is an essential co-factor for the transformation of kermesic acid to carminic acid.

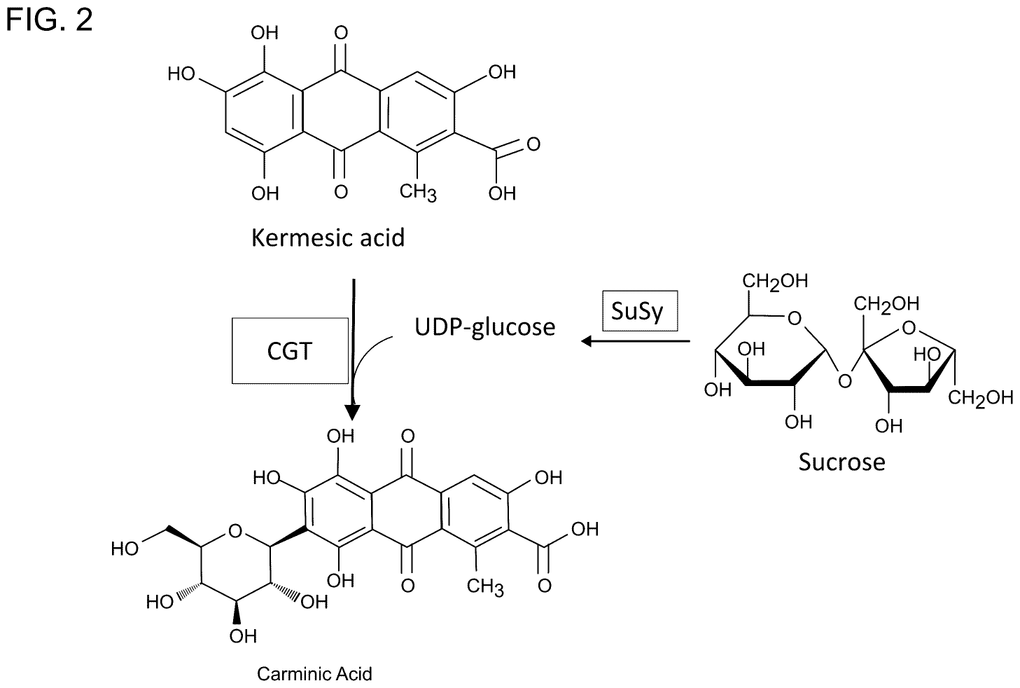

In certain aspects, the activated sugar and/or UDP-glucose is added to the reaction medium. In certain embodiments, the activated sugar in the reaction medium for cell-free production of carminic acid is UDP-glucose. In certain embodiments, the UDP-glucose for the reaction medium is generated from sucrose. In certain embodiments, the UDP-glucose is generated in the reaction medium by one or more enzymes. In certain embodiments, the generation of UDP-sugar is mediated by sucrose synthase (SuSy). In certain embodiments, sucrose synthase (SuSy) mediates the conversion of sucrose to UDP-glucose. The SuSy mediated conversion of sucrose to UDP-glucose is schematically described in FIG. 2. As demonstrated in FIG. 2, SuSy is involved in the conversion of sucrose to UDP-glucose, and the produced UDP-glucose is involved in the conversion of kermesic acid to carminic acid. Thus, in certain embodiments, the reaction medium comprises sucrose, which is subsequently converted to UDP-glucose in course of the reaction. In certain embodiments, the conversion of the sucrose to UDP-glucose may be carried out in the same reactor as the conversion of kermesic acid to carminic acid. In other embodiments, the conversion of the sucrose to UDP-glucose may be carried out in a different reactor as the conversion of kermesic acid to carminic acid.

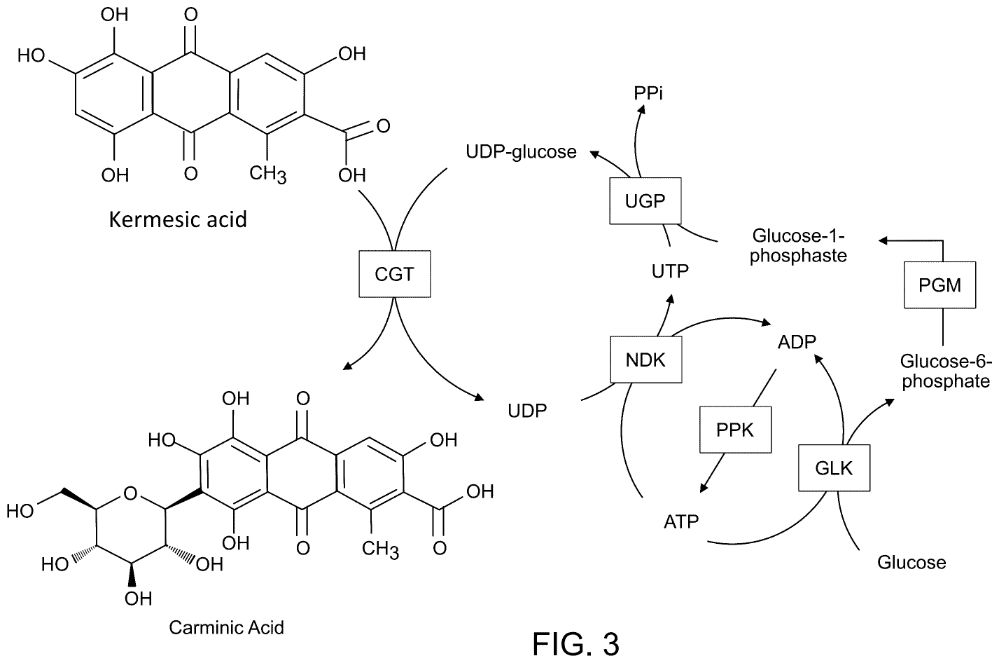

In certain beneficial aspects, the invention provides that the UDP-glucose used in the reaction is generated from other substrates in course of the reaction. In certain embodiments, the UDP moiety in UDP-glucose is recycled in course of the process. The recycling of UDP provides economic efficiency of the processes of the invention. Accordingly, in certain aspects, the production of carminic acid from kermesic acid catalyzed by CGT is conducted in conjunction with other methods for UDP-glucose production or recycling of UDP. In certain embodiments, the overview of the synthetic scheme involving the UDP-glucose production and/or recycling is provided in FIG. 3. In certain embodiments, UDP-glucose is generated by the reaction of uridine triphosphate (UTP) with glucose-1-phosphate. In certain preferred embodiments, the reaction of UTP and glucose-1-phosphate is catalyzed by UTP-glucose-1-phosphate uridylyltransferase (UGP). In certain preferred embodiments, the UGP may be galU.

In certain aspects, glucose-1-phosphate is the source for formation of UDP-glucose. Accordingly, in certain embodiments, glucose-1-phosphate may be added to the reaction for generation of carminic acid. In certain embodiments, the glucose-1-phosphate may be supplied to the reaction for preparation of carminic acid.

In certain embodiments, glucose-1-phosphate is generated during the course of the reaction. In certain embodiments, glucose-1-phosphate is generated enzymatically during the course of the reaction. Accordingly, in certain embodiments, glucose is converted to glucose-6-phosphate by a reaction of ATP (adenosine triphosphate) and glucose. In certain embodiments, reaction between glucose and ATP results in generation of glucose-6-phosphate and adenosine diphosphate (ADP). In certain embodiments, conversion of glucose to glucose-6-phosphate is catalyzed by glucokinase (GLK). In certain embodiments, conversion of glucose to glucose-6-phosphate is catalyzed by hexokinase (HK). In certain embodiments, glucose-6-phosphate is converted to glucose-1-phosphate. In certain embodiments, the conversion of glucose-6-phosphate to glucose-1-phsophate is catalyzed by phosphoglucomutase (PGM).

In certain aspects, the invention further provides that the nucleoside triphosphate species used in the reaction are recycled. In certain embodiments, the recycled nucleoside trisphosphates are ATP and UTP. In certain embodiments, UTP is generated by reaction of UDP and ATP. In certain embodiments, the generation of UTP from UDP and ATP is catalyzed by nucleoside diphosphate kinase (NDK). In certain embodiments, ATP may be generated from ADP and phosphate or polyphosphate. In certain embodiments, the conversion of ADP to ATP is catalyzed by polyphosphate kinase (PPK).

In certain beneficial aspects, the invention provides that the processes of the invention are cost effective because of the recycling of chemical intermediates involved in the reaction. In certain embodiments, the invention provides that chemical intermediates like UDP-glucose, UDP, and glucose-1-phosphate are recycled. Indeed, in certain embodiments, the only reagents required in non-catalytic quantities for the production of carminic acid by conversion of kermesic acid are polyphosphate/phosphate and glucose. Because the processes provided in the invention are conducted in a cell free medium and the ingredients (required in non-catalytic quantities) are economic, the processes of the invention are cost effective.

In certain aspects, the one or more enzymes required for the cell-free production of carminic acid are expressed in a host organism. In certain embodiments, the host organism is selected from a group consisting of: bacteria, yeast, and/or mammalian cells. In certain embodiments, the one or more enzymes are introduced in the host organism by integration into genome of the host organism or on a plasmid. In certain embodiments, the plasmid comprises extrachromosomal DNA, which can be expressed by the host organism. In certain embodiments, host organisms expressing the one or more enzymes are cultured until a pre-determined biomass is achieved to produce the requisite quantity of the one or more enzymes. The predetermined biomass is calculated based on the quantity of the one or more enzymes required for the cell-free production of carminic acid. In certain embodiments, the culture comprising host organisms expressing the one or more enzymes are lysed and used as the reaction medium for the methods provided in the invention. In certain other embodiments, once the culture comprising host organisms is lysed, the cell-debris is removed from the lysed matter to prepare the reaction medium for the methods of the invention. In certain embodiments, the one or more enzymes expressed in the host cells are C-glucosyltransferase (CGT) and/or sucrose synthase (SuSy). In certain embodiments, C-glucosyltransferase (CGT) and/or sucrose synthase (SuSy) are present in the cell-free medium for the cell-free production of carminic acid.

In certain embodiments of the invention, the reaction mixture comprises purified enzymes for the cell-free production of carminic acid. In certain embodiments, the purified enzymes are C-glucosyltransferase (CGT) and/or sucrose synthase (SuSy).

In certain embodiments, the one or more enzymes expressed in host cells are C-glucosyltransferase (CGT), sucrose synthase (SuSy), glucokinase (GLK), hexokinase (HK), phosphoglucomutase (PGM), polyphosphate kinase (PPK), UTP-glucose-1-phosphate uridylyltransferase (UGP), and/or nucleoside diphosphate kinase (NDK). In certain embodiments, C-glucosyltransferase (CGT), sucrose synthase (SuSy), glucokinase (GLK), hexokinase (HK), phosphoglucomutase (PGM), polyphosphate kinase (PPK), UTP-glucose-1-phosphate uridylyltransferase (UGP), and/or nucleoside diphosphate kinase (NDK) are present in the cell-free medium for the cell-free production of carminic acid.

In certain embodiments of the invention, the reaction mixture comprises purified enzymes for the cell-free production of carminic acid. In certain embodiments, the purified enzymes are C-glucosyltransferase (CGT), sucrose synthase (SuSy), glucokinase (GLK), hexokinase (HK), phosphoglucomutase (PGM), polyphosphate kinase (PPK), UTP-glucose-1-phosphate uridylyltransferase (UGP), and/or nucleoside diphosphate kinase (NDK). In certain embodiments, C-glucosyltransferase (CGT), sucrose synthase (SuSy), glucokinase (GLK), hexokinase (HK), phosphoglucomutase (PGM), polyphosphate kinase (PPK), UTP-glucose-1-phosphate uridylyltransferase (UGP), and/or nucleoside diphosphate kinase (NDK). In certain embodiments, the purified enzymes are C-glucosyltransferase (CGT) and/or sucrose synthase (SuSy).

In certain embodiments, the one or more enzymes to produce carminic acid is glucosyltransferase (GT) and/or C-glucosyltransferase (CGT). C-Glycosyltransferases (CGT) catalyze the formation of C-glycosidic bonds for the biosynthesis of C-glycosides. Glycosylation occurs on O-, C-, N-, and S-atoms to produce O-glycosides, C-glycosides, N-glycosides, and S-glycosides, respectively. C-Glycosides are more stable in response to acid hydrolysis and glycosidase than the others. The chemical synthesis of C-glycosides is always challenging due to stereoselectivity and harsh reaction conditions. The difficulty in preparing sugar precursors also restricts the synthesis of C-glycosides. By contrast, the biosynthesis of glycosides involves much milder conditions and has high stereospecificity. Therefore, research on C-glycosyltransferases (CGTs) is beneficial for studying the biosynthesis of C-glycosides, thereby facilitating the development and utilization of C-glycoside-based drugs.

The previously known enzymes for this process for this process of production of carminic acid was from the cochineal beetle Dactylopius coccus (DcUGT) and an engineered variant from the plant Gentiana trifloral (GtCGT). In certain embodiments, the invention provides an improved GT enzyme engineered to change product specificity as a CGT instead of an OGT.

In certain embodiments, the GT and/or CGT used for cell-free production of carminic acid are provided in WO 2022/164226, WO 2022/013659, and/or U.S. Pat. No. 10,724,012, which are incorporated by reference herein in their entirety.

In certain embodiments, GT and/or CGT used for cell-free production of carminic acid is selected from the enzymes having at least 80% amino acid sequence identity from the enzymes provided in Table 1 below. In certain embodiments, GT and/or CGT used for cell-free production of carminic acid is selected from the enzymes having at least 95% amino acid sequence identity from the enzymes provided in Table 1 (reproduced below).

TABLE 1

| Accession # | Organism |
|---|---|
| AXF38108 | Bergenia purpurascens |
| AFJ52990 | Linum usitatissimum |
| KAG8368435 | Buddleja alternifolia |
| AFJ52991 | Linum usitatissimum |
| AUI41123 | Rhodiola rosea |
| KAD6795309 | Mikania micrantha |
| XP_006282251 | Capsella rubella |
| BAF18172 | Oryza sativa Japonica Group |
| A0A0B6VIJ5 | Gentiana trifloral |
| GKV43195.1 | Shorea leprosula |
| CAA7037192.1 | Microthlaspi erraticum |
| CAD6239704.1 | Miscanthus lutarioriparius |
| XP_021750757 | Chenopodium quinoa |
| BAG14302 | Gomphrena globose |
| XP_031382117 | Punica granatum |

In certain embodiments, the one or more enzymes involved in production of UDP-glucose, which is an ingredient in the cell-free production of carminic acid is sucrose synthase (SuSy). Sucrose synthase (SuSy) converts sucrose and uridine 5′-diphosphate (UDP) into UDP-glucose. Coupling of SuSy and GT reactions in one-pot cascade transformations creates a UDP cycle, which regenerates the UDP-glucose continuously and so makes it an expedient donor for glucoside production. In certain embodiments, SuSy used for production of UDP-glucose is selected from the enzymes having at least 80% amino acid sequence identity from the enzymes provided in Table 2 below. In certain embodiments, SuSy used for production of UDP-glucose is selected from the enzymes having at least 95% amino acid sequence identity from the enzymes provided in Table 2 (reproduced below).

TABLE 2

| Accession # | Organism |
|---|---|
| AXF38108 | Bergenia purpurascens |
| AFJ52990 | Linum usitatissimum |
| KAG8368435 | Buddleja alternifolia |
| AFJ52991 | Linum usitatissimum |
| AUI41123 | Rhodiola rosea |
| KAD6795309 | Mikania micrantha |
| XP_006282251 | Capsella rubella |
| BAF18172 | Oryza sativa Japonica Group |
| A0A0B6VIJ5 | Gentiana trifloral |
| GKV43195.1 | Shorea leprosula |
| CAA7037192.1 | Microthlaspi erraticum |
| CAD6239704.1 | Miscanthus lutarioriparius |
| XP_021750757 | Chenopodium quinoa |
| BAG14302 | Gomphrena globose |
| XP_031382117 | Punica granatum |

In certain embodiments, the one or more enzymes involved in generation of UDP-glucose and/or recycling of one or more reaction intermediates are provided below. In certain embodiments, the enzymes used for generation of UDP-glucose (by reaction with UTP) and/or recycling of one or more intermediates are selected from the enzymes having at least 95% amino acid sequence identity from the enzymes provided in Table 3 below.

TABLE 3

| Enzyme | Accession No. |
|---|---|
| GLK | AAA27694.1 |
| GLK | WP_331012003.1 |
| PGM | AAC73782.1 |
| PGM | A0AI10EI47 |
| PGM | A0A7M2S2U2 |
| UGP | EEW2752841.1 |
| UGP | WP_021648042.1 |
| UGP | WP_028847555.1 |
| NDK | WP_000963837.1 |
| NDK | CAF21035.1 |
| NDK | WP_066795798 |
| PPK2 | WP_010968631 |
| PPK2 | WP_160857702 |

In certain embodiments, the invention provides engineered GT/CGT/SuSy enzymes for the cell-free production of carminic acid. In certain embodiments, the engineered GT/CGT/SuSy enzymes were optimized for cell-free production of carminic acid. In certain embodiments, engineered GT/CGT/SuSy enzymes may include genetic modifications. In certain embodiments, the genetic modifications may be selected from a group consisting of: point mutations, insertions, deletions, and/or any other modifications such that those enzymes result in efficient and optimal cell-free production of carminic acid. In certain embodiments, the GT/CGT/SuSy enzymes used in the cell-free production of carminic acid are any enzymes disclosed in this application.

In certain embodiments, the invention provides engineered and optimized GLK/PGM/UGP/NDK/PPK enzymes for generation of UDP-glucose and/or recycling of reaction intermediates involved in cell-free production of carminic acid. In certain embodiments, the engineered and/or modified GLK/PGM/UGP/NDK/PPK enzymes may include genetic modifications. In certain embodiments, the genetic modifications may be selected from a group consisting of: point mutations, insertions, deletions, and/or any other modifications such that those enzymes result in efficient and optimal cell-free production of carminic acid.

In certain aspects of the invention, the method of the cell-free production does not require the purification of the one or more enzymes from the lysed host organisms. In certain embodiments, the methods of the invention do not require the purification of one or more enzymes from the lysed biomass comprising host cell expressing the one or more enzymes for the cell-free production of carminic acid. In certain embodiments, the methods of the invention do not require the purification of CGT/SuSy/GLK/PGM/UGP/NDK/PPK from the lysed cells for cell-free production of carminic acid. This is advantageous because it increases the efficiency of the method and reduces the costs associated with the purification of these enzymes.

In certain embodiments, the cell-free medium may further comprise any other additional ingredients required for the cell-free production of carminic acid. For example, in certain embodiments, the cell-free medium comprises buffer, kermesic acid, an activated sugar, magnesium chloride, cell lysate, sucrose, and/or water. In certain embodiments, the cell-free reaction mixture further comprises uridine diphosphate (UDP). In certain embodiments, the cell-free reaction mixture further comprises polyphosphate and/or glucose. In certain embodiments, the cell-free reaction mixture comprises UDP-glucose and/or UDP in catalytic quantities. In certain embodiments, UDP-glucose is produced enzymatically in the reaction. In certain embodiments, the reaction mixture comprises glucose-1-phosphate. Glucose-1-phosphate may be generated in the cell-free reaction. Thus, in certain embodiments, glucose-1-phosphate is also present in catalytic quantity. In certain embodiments, the reaction medium comprises glucose and polyphosphate.

In certain preferred embodiments, the processes of the invention utilize kermesic acid, glucose, and polyphosphate as the starting materials for production of carminic acid. In these embodiments, the invention provides that the other intermediates in the process for production of carminic acid may be recycled.

In certain embodiments, the processes of the invention utilize kermesic acid and UDP-glucose as the starting materials for production of carminic acid.

In certain embodiments, the processes of the invention utilize kermesic acid and sucrose as the starting materials for production of carminic acid. In these embodiments, the invention provides that the other intermediates in the process for production of carminic acid may be recycled.

In certain embodiments, the buffer used in the cell-free reaction medium is any buffer suitable for enzymatic conversion of kermesic acid to carminic acid. In certain embodiments, the buffer maintains the pH of about 5 to about 9 in the reaction mixture. In certain embodiments, the buffer maintains the pH of about 6 to about 8 in the reaction mixture. In certain embodiments, the buffer maintains the pH in the range of 6 to 8 in the reaction mixture.

In certain embodiments, the buffer is selected from a group consisting of: Tris, HEPES, phosphate, carbonate/bicarbonate, acetate, sodium hydroxide-glycine, and/or citrate. The concentration of the buffer for the reaction mixture would depend on the concentration of the starting materials and other ingredients in the reaction mixture. In certain embodiments, the buffer is a phosphate buffer. In certain embodiments, the buffer is present at a concentration of about 1 mM to about 250 mM. In certain embodiments, the buffer is present at a concentration of about 5 mM to about 100 mM. In certain embodiments, the phosphate buffer is present at a concentration of about 1 mM to about 250 mM. In certain embodiments, the phosphate buffer is present at a concentration of about 5 mM to about 100 mM.

In certain embodiments, the cell-free reaction medium comprises activated sugar at a concentration of about 0.001 mM to about 50 mM. In certain embodiments, the cell-free reaction medium comprises activated sugar at a concentration of about 0.01 mM to about 5 mM. In certain embodiments, the activated sugar is UDP-glucose.

In certain embodiments, the cell-free reaction medium comprises a source of magnesium ions. In certain embodiments, the source of magnesium ions is magnesium chloride at a concentration of about 0.5 mM to about 40 mM. In certain embodiments, the cell-free reaction medium comprises magnesium chloride at a concentration of about 1 mM to about 20 mM.

In certain embodiments, the cell-free reaction medium further comprises sucrose. In certain embodiments, the sucrose is converted to UDP-glucose. Thus, the concentration of sucrose in the reaction mixture is determined by the quantity of UDP-glucose needed for the reaction for cell-free production of carminic acid. In certain embodiments, sucrose is present at a concentration of about 10 mM to about 1000 mM in the cell-free reaction medium. In certain embodiments, sucrose is present at a concentration of about 20 mM to about 800 mM in the cell-free reaction medium. In certain embodiments, sucrose is present at a concentration of about 50 mM to about 600 mM in the cell-free reaction medium.

In certain embodiments, the cell-free reaction medium further comprises glucose. In certain embodiments, glucose is a starting material to provide UDP-glucose for the conversion of kermesic acid to carminic acid. Thus, the concentration of glucose in the reaction mixture is determined by the quantity of UDP-glucose needed for the reaction for cell-free production of carminic acid. In certain embodiments, glucose is present at a concentration of about 10 mM to about 1000 mM in the cell-free reaction medium. In certain embodiments, glucose is present at a concentration of about 20 mM to about 800 mM in the cell-free reaction medium. In certain embodiments, glucose is present at a concentration of about 50 mM to about 600 mM in the cell-free reaction medium.

In certain embodiments, the cell-free reaction medium further comprises polyphosphate. In certain embodiments, polyphosphate is also a starting material to provide UDP-glucose for the conversion of kermesic acid to carminic acid. Thus, the concentration of polyphosphate in the reaction mixture is determined by the quantity of UDP-glucose needed for the reaction for cell-free production of carminic acid. In certain embodiments, polyphosphate is present at a concentration of about 10 mM to about 1000 mM in the cell-free reaction medium. In certain embodiments, polyphosphate is present at a concentration of about 20 mM to about 800 mM in the cell-free reaction medium. In certain embodiments, polyphosphate is present at a concentration of about 50 mM to about 600 mM in the cell-free reaction medium.

In certain embodiments, the quantity of the one or more enzymes for the cell-free production of carminic acid is dependent on the target quantity of carminic acid to be produced and/or the concentration of other ingredients present in the reaction mixture. In certain embodiments, the one or more enzymes are present in a concentration of from about 1% to about 50% (v/v). In certain embodiments, the one or more enzymes are present in a concentration of from about 2.5% to about 45% (v/v). In certain embodiments, the one or more enzymes are present in a concentration of from about 5% to about 40% (v/v). In certain embodiments, the one or more enzymes are present in a concentration of from about 7.5% to about 30% (v/v).

In certain embodiments, the reaction for production of carminic acid is conducted for a duration till the desired quantity of carminic acid is obtained. In certain embodiments, the reaction for cell-free production of carminic acid is carried out from about 10 minutes to about 48 hours. In certain embodiments, the reaction for cell-free production of carminic acid is carried out from about 10 minutes to about 36 hours. In certain embodiments, the reaction for cell-free production of carminic acid is carried out from about 20 minutes to about 24 hours. In certain embodiments, the reaction for cell-free production of carminic acid is carried out from about 30 minutes to about 20 hours. In certain embodiments, the reaction for cell-free production of carminic acid is carried out from about 1 hour to about 15 hours.

In certain embodiments, the temperature of the reaction mixture for cell-free production of carminic acid is varied to obtain optimal results for the production of carminic acid. In certain embodiments, the temperature of the reaction mixture for cell-free production of carminic acid is from about 15° C. to about 45° C. In certain embodiments, the temperature of the reaction mixture for cell-free production of carminic acid is from about 20° C. to about 40° C. In certain embodiments, the temperature of the reaction mixture for cell-free production of carminic acid is from about 22.5° C. to about 37.5° C. The temperature of the reaction mixture for cell-free production of carminic acid may influence the rate of production of carminic acid. Consequently, the duration of reaction may be adjusted according to the temperature of the reaction mixture to obtain optimal yield of carminic acid in cell-free production of carminic acid.

In certain aspects, the methods provided in the invention may be carried out in any reactor suitable for carrying out the cell-free production of carminic acid. In certain embodiments, the reaction for cell-free production of carminic acid is conducted in a bubble column reactor/bioreactor. In certain embodiments, in the bubble column reactor/bioreactor, the one or more enzymes involved in cell-free production of carminic acid are in a solution. In certain embodiments, the reaction for cell-free production of carminic acid is conducted in a bubble column reactor/bioreactor comprises the lysate from the host organism. In certain embodiments, it is advantageous to use the bubble column reactor/bioreactor for cell-free production of carminic acid when the reaction mixture involves the lysate (or lysate with cellular debris removed) from the host cell organisms in which the one or more enzymes responsible for cell-free production of carminic acid were utilized. In certain embodiments, the reaction for cell-free production of carminic acid is conducted in a packed bed reactor/bioreactor. In certain embodiments, the one or more enzymes are immobilized in the packed bed reactor/bioreactor. The packed bed reactors/bioreactors are preferred for the purified enzymes playing a role in cell-free production of carminic acid. In certain embodiments, the one or more enzymes may be immobilized in a single reactor/bioreactor. In certain other embodiments, the one or more enzymes may be immobilized in different reactors/bioreactors, wherein these reactors/bioreactors are linked sequentially. In certain embodiments, the bioreactor system provided in PCT/US2021/064049, incorporated by reference in its entirety.

The methods provided in the invention are advantageous over other conventional methods of production of carminic acid. In certain embodiments, the methods of the invention provide cell-free production of carminic acid. Because the methods of the invention are conducted in cell-free medium, they provide significant economic efficiency by reducing the cost of production of carminic acid in other conventional methods. In certain embodiments, because the reaction for production of carminic acid is conducted from the lysates from the host organisms expressing the one or more enzymes involved in the reaction, the methods of the invention are cost-efficient. In particular, in certain embodiments, the methods of the invention do not involve the purification of the one or more enzymes. Because purification of individual enzymes is not required in the methods of the invention, provides further economic efficiency by reducing the cost that would have been otherwise required in purifying individual enzymes.

Advantageously, the cell-free production of carminic acid provided herein provides a significantly higher titer value for carminic acid as compared to the conventional methods. The higher titer values of carminic acid provide additional cost advantages for production of carminic acid because the higher titer carminic acid would provide efficiency in purifying and/or concentrating carminic acid from the reaction mixture. In certain embodiments, the methods of the invention provide carminic acid titer value of from at least 5-fold higher than the conventional methods. In certain embodiments, the methods of the invention provide carminic acid titer value of from at least 10-fold higher than the conventional methods. In certain embodiments, the methods of the invention provide carminic acid titer value of from at least 50-fold higher than the conventional methods. In certain embodiments, the methods of the invention provide carminic acid titer value of from at least 100-fold higher than the conventional methods. In certain embodiments, the methods of the invention provide carminic acid titer value of from at least 500-fold higher than the conventional methods. In certain embodiments, the methods of the invention provide carminic acid titer value of from at least 1000-fold higher than the conventional methods. In certain embodiments, the methods of the invention provide carminic acid titer value of from at least 5000-fold higher than the conventional methods.

In some embodiments, the isolated carminic acid has a purity of about 10%, or about 20%, or about 30%, or about 40%, or about 50%, or about 60%, or about 70%, or about 80%, or about 90%, or about 95%, or about 99%, or about 100%.

In other embodiments, the isolated carminic acid has a purity of from about 10% to 95%, or from about 10% to 90%, or from about 10% to 80% or from about 10% to 70%, or from about 10% to 60%, or from about 10% to 50%, or from about 10% to 40%, or from about 20% to 95%, or from about 20% to 90%, or from about 20% to 80% or from about 20% to 70%, or from about 20% to 60%, or from about 20% to 50%, or from about 20% to 40%, or from about 50% to 95%, or from about 50% to 90%, or from about 50% to 80% or from about 50% to 70%, or from about 50% to 60%.

### Compositions for Cell-Free Production of Carminic Acid

In certain aspects, the invention provides compositions for cell-free production of carminic acid. The compositions of the invention are utilized for cell-free production of carminic acid in accordance with the methods described above.

In certain embodiments, the compositions of the invention comprise: one or more enzymes in a cell-free medium, wherein the one or more enzymes result in transformation of a substrate to carminic acid. In certain embodiments, the substrate converted to carminic acid is kermesic acid. In certain embodiments, the one or more enzyme is C-glucosyltransferase (CGT). In certain embodiments, the compositions further comprise a cell-free medium. In certain embodiments, the cell-free medium comprises an activated sugar. In certain embodiments, the activated sugar is UDP-glucose. In certain embodiments, the UDP-glucose is added to the cell-free medium.

In certain aspects of the invention, the UDP-glucose in the composition is synthesized in the cell-free medium by the one or more enzymes. In certain embodiments, the UDP-glucose is synthesized from sucrose. In certain embodiments, the one or more enzymes is sucrose synthase (SuSy) that synthesizes UDP-glucose from sucrose. In certain embodiments, the cell-free medium is a cell lysate. In certain embodiments, the cell lysate is a cell lysate from cells from a host organism expressing the one or more enzymes. In certain embodiments, the host organism is selected from a group consisting of: bacteria, yeast, and/or mammalian cells. In certain embodiments, the one or more enzymes are introduced in the host organism by integration into genome of the host organism or on a plasmid. In certain embodiments, the host organisms expressing the one or more enzymes are cultured until a pre-determined biomass is achieved to produce the requisite quantity of the one or more enzymes. In certain embodiments, the composition further comprises cell lysate generated by lysis of cells and subsequent removal of cell debris, for use in the cell-free medium for cell-free production of carminic acid. In certain embodiments, the CGT and SuSy enzymes are present in the cell-free medium for cell-free production of carminic acid. In certain embodiments, the compositions of the invention provide CGT and SuSy that have not been purified or separated.

In certain embodiments, the composition further comprises glucose-1-phosphate. In certain embodiments, glucose-1-phosphate is the source for formation of UDP-glucose. Accordingly, in certain embodiments, glucose-1-phosphate may be added to the reaction for generation of carminic acid. In certain embodiments, glucose-1-phosphate may be supplied to the reaction for preparation of carminic acid.

In certain embodiments, in the compositions of the invention, glucose-1-phosphate is generated during the course of the reaction. In certain embodiments, glucose-1-phosphate is generated enzymatically during the course of the reaction. Accordingly, in certain embodiments, the compositions of the invention comprise glucose and/or ATP. In certain embodiments, glucose is converted to glucose-6-phosphate by a reaction of ATP and glucose. In certain embodiments, reaction between glucose and ATP results in generation of glucose-6-phosphate and adenosine diphosphate (ADP). In certain embodiments, conversion of glucose to glucose-6-phosphate is catalyzed by glucokinase (GLK). In certain embodiments, conversion of glucose to glucose-6-phosphate is catalyzed by hexokinase (HK). In certain embodiments, glucose-6-phosphate is converted to glucose-1-phosphate. In certain embodiments, the conversion of glucose-6-phosphate to glucose-1-phsophate is catalyzed by phosphoglucomutase (PGM). Accordingly, in certain embodiments, the composition comprises GLK, HK, and/or PGM.

In certain aspects, the invention further provides that the nucleoside triphosphate species used in the reaction are recycled. In certain embodiments, the recycled nucleoside trisphosphates are ATP and UTP. In certain embodiments, UTP is generated by reaction of UDP and ATP. In certain embodiments, the generation of UTP from UDP and ATP is catalyzed by nucleoside diphosphate kinase (NDK). In certain embodiments, ATP may be generated from ADP and phosphate or polyphosphate. In certain embodiments, the conversion of ADP to ATP is catalyzed by polyphosphate kinase (PPK). In certain embodiments, the compositions of the invention further comprise ATP, UTP, and/or UDP. In certain embodiments, the compositions of the invention further comprise PPK.

In certain embodiments, the composition further comprises C-glucosyltransferase (CGT), sucrose synthase (SuSy), glucokinase (GLK), hexokinase (HK), phosphoglucomutase (PGM), polyphosphate kinase (PPK), UTP-glucose-1-phosphate uridylyltransferase (UGP), and/or nucleoside diphosphate kinase (NDK). In certain embodiments, C-glucosyltransferase (CGT), sucrose synthase (SuSy), glucokinase (GLK), hexokinase (HK), phosphoglucomutase (PGM), polyphosphate kinase (PPK), UTP-glucose-1-phosphate uridylyltransferase (UGP), and/or nucleoside diphosphate kinase (NDK) are present in the cell-free medium for the cell-free production of carminic acid.

In certain embodiments, the cell-free medium may further comprise any other additional ingredients required for the cell-free production of carminic acid. For example, in certain embodiments, the cell-free medium comprises buffer, kermesic acid, an activated sugar, magnesium chloride, cell lysate, sucrose, and/or water. In certain embodiments, the buffer used in the cell-free reaction medium is any buffer suitable for enzymatic conversion of kermesic acid to carminic acid. In certain embodiments, the buffer maintains the pH of about 5 to about 9 in the reaction mixture. In certain embodiments, the buffer maintains the pH of about 6 to about 8 in the reaction mixture. In certain embodiments, the buffer maintains the pH in the range of 6 to 8 in the reaction mixture. In certain embodiments, the buffer is a phosphate buffer. In certain embodiments, the buffer is present at a concentration of about 1 mM to about 200 mM. In certain embodiments, the buffer is present at a concentration of about 5 mM to about 100 mM.

In certain embodiments, the cell-free reaction medium comprises activated sugar at a concentration of about 0.001 mM to about 50 mM. In certain embodiments, the cell-free reaction medium comprises activated sugar at a concentration of about 0.01 mM to about 5 mM. In certain embodiments, the activated sugar is UDP-glucose.

In certain embodiments, the cell-free reaction medium comprises magnesium chloride at a concentration of about 0.5 mM to about 40 mM. In certain embodiments, the cell-free reaction medium comprises magnesium chloride at a concentration of about 1 mM to about 20 mM.

In certain embodiments, the cell-free reaction medium further comprises sucrose. In certain embodiments, the sucrose is converted to UDP-glucose. Thus, the concentration of sucrose in the reaction mixture is determined by the quantity of UDP-glucose needed for the reaction for cell-free production of carminic acid. In certain embodiments, sucrose is present at a concentration of about 10 mM to about 1000 mM in the cell-free reaction medium. In certain embodiments, sucrose is present at a concentration of about 20 mM to about 800 mM in the cell-free reaction medium. In certain embodiments, sucrose is present at a concentration of about 50 mM to about 600 mM in the cell-free reaction medium.

In certain embodiments, the cell-free reaction medium further comprises glucose. In certain embodiments, the concentration of glucose in the reaction mixture is determined by the quantity of UDP-glucose needed for the reaction for cell-free production of carminic acid. In certain embodiments, glucose is present at a concentration of about 1 mM to about 1000 mM in the cell-free reaction medium. In certain embodiments, glucose is present at a concentration of about 10 mM to about 1000 mM in the cell-free reaction medium. In certain embodiments, glucose is present at a concentration of about 50 mM to about 600 mM in the cell-free reaction medium.

In certain embodiments, the cell-free reaction medium further comprises phosphate/polyphosphate. In certain embodiments, the cell-free reaction medium further comprises polyphosphate. In certain embodiments, the concentration of polyphosphate in the reaction mixture is determined by the quantity of UDP-glucose needed for the reaction for cell-free production of carminic acid. In certain embodiments, polyphosphate is present at a concentration of about 0.1 g/L to about 500 g/L in the cell-free reaction medium. In certain embodiments, polyphosphate is present at a concentration of about 1 g/L to about 100 g/L in the cell-free reaction medium. In certain embodiments, polyphosphate is present at a concentration of about 1 g/L to about 60 g/L in the cell-free reaction medium. In certain embodiments, polyphosphate is present at a concentration of about 5 g/L to about 40 g/L in the cell-free reaction medium. In certain embodiments, polyphosphate is present at a concentration of about 25 g/L.

In certain embodiments, the cell-free reaction medium further comprises UTP and/or UDP. In certain embodiments, the concentration of UTP and/or UDP in the reaction mixture is determined by the quantity of UDP-glucose needed for the reaction for cell-free production of carminic acid. In certain embodiments, UTP and/or UDP is in the concentration of about 0.01 M to about 100 μM in the cell-free medium. In certain embodiments, UTP and/or UDP is in the concentration of about 0.1 μM to about 50 M in the cell-free medium. In certain embodiments, UTP and/or UDP is in the concentration of about 0.1 μM to about 10 UM in the cell-free medium. In certain embodiments, UTP and/or UDP is in the concentration of about 1 μM in the cell-free medium. In certain embodiments, UTP and/or UDP is in the concentration of about 5 μM in the cell-free medium.

In certain embodiments, the cell-free reaction medium further comprises ATP. In certain embodiments, the concentration of ATP in the reaction mixture is determined by the quantity of UDP-glucose needed for the reaction for cell-free production of carminic acid. In certain embodiments, ATP is in the concentration of about 0.01 μM to about 100 μM in the cell-free medium. In certain embodiments, ATP is in the concentration of about 0.1 μM to about 50 μM in the cell-free medium. In certain embodiments, ATP is in the concentration of about 0.1 μM to about 10 μM in the cell-free medium. In certain embodiments, ATP is in the concentration of about 1 μM in the cell-free medium. In certain embodiments, ATP is in the concentration of about 5 μM in the cell-free medium.

In certain embodiments, the cell-free reaction medium comprises glucose-6-phosphate and/or glucose-1-phosphate. In certain embodiments, glucose-6-phosphate and/or glucose-1-phosphate are generated and/or recycled in the reaction medium. Thus, in certain embodiments, glucose-6-phosphate and/or glucose-1-phosphate are present in catalytic quantities in the reaction medium. In certain embodiments, glucose-6-phosphate and/or glucose-1-phosphate may be added in the reaction medium. In certain embodiments, glucose-6-phosphate and/or glucose-1-phosphate are present at a concentration of from about 0.01 mM to about 500 mM. In certain embodiments, glucose-6-phosphate and/or glucose-1-phosphate are present at a concentration of from about 0.01 mM to about 1 mM. In certain embodiments, glucose-6-phosphate and/or glucose-1-phosphate are present at a concentration of from about 1 mM to about 500 mM. In certain embodiments, glucose-6-phosphate and/or glucose-1-phosphate are present at a concentration of from about 10 mM to about 500 mM. In certain embodiments, glucose-6-phosphate and/or glucose-1-phosphate are present at a concentration of from about 10 mM to about 100 mM.

In certain embodiments, the quantity of the one or more enzymes for the cell-free production of carminic acid is dependent on the target quantity of carminic acid to be produced and/or the concentration of other ingredients present in the reaction mixture. In certain embodiments, the one or more enzymes are present in a concentration of from about 1% to about 50% (v/v). In certain embodiments, the one or more enzymes are present in a concentration of from about 2.5% to about 45% (v/v). In certain embodiments, the one or more enzymes are present in a concentration of from about 5% to about 40% (v/v). In certain embodiments, the one or more enzymes are present in a concentration of from about 7.5% to about 30% (v/v).

In certain aspects of the invention, the compositions of the invention are in a bubble column reactor, wherein the one or more enzymes are in a solution. In certain embodiments, the compositions of the invention are in a packed bed reactor, wherein the one or more enzymes are immobilized.

In some embodiments, the enzymes may be immobilized. In some embodiments, immobilized enzymes may be immobilized onto solid supports. Non-limiting examples of solid supports may include (but are not limited to) epoxy methacrylate, carboxymethyl-cellulose, starch, collagen, ion exchange resins, amino C₆ methacrylate, or microporous polymethacrylate. In further embodiments, various surface chemistries may be used for linking the immobilized enzyme to a solid surface, including but not limited to covalent, adsorption, ionic, affinity, encapsulation, or entrapment. In other embodiments, immobilized enzymes may be immobilized in crosslinked enzyme aggregates. In other embodiments, the enzymes are non-immobilized. Either immobilized or non-immobilized enzymes may be employed in batch or continuous synthesis. For example, an immobilized enzyme on a solid support may be used in a cartridge through which a reaction mixture passes, whereby an immobilized enzyme may catalyze modification of substrate to produce the product at a high titer. Alternatively, a continuous method may comprise micro mixing of enzyme solution and substrate to produce the product at a high titer, while continuously removing product, removing (e.g., recovering) substrate, or both. In some embodiments removed (e.g., recovered) substrate may be recycled to increase process efficiency and overall yield.

In some embodiments, C-glucosyltransferase (CGT), sucrose synthase (SuSy), glucokinase (GLK), hexokinase (HK), phosphoglucomutase (PGM), polyphosphate kinase (PPK), UTP-glucose-1-phosphate uridylyltransferase (UGP), and/or nucleoside diphosphate kinase (NDK) are immobilized.

In some embodiments, C-glucosyltransferase (CGT), sucrose synthase (SuSy), glucokinase (GLK), hexokinase (HK), phosphoglucomutase (PGM), polyphosphate kinase (PPK), UTP-glucose-1-phosphate uridylyltransferase (UGP), and/or nucleoside diphosphate kinase (NDK) are non-immobilized.

In some embodiments, enzymes are recycled by ultrafiltration. In some embodiments ion exchange resins may be used to capture carminic acid during production. For example, amine-functionalized solid support may be added to capture carminic acid for continuous purification from reaction mixture. In certain embodiments, the bioreactor system provided in PCT/US2021/064049, incorporated by reference in its entirety.

Advantageously, the cell-free production of carminic acid provided herein provides a significantly higher titer value for carminic acid as compared to the conventional methods. The higher titer values of carminic acid provide additional cost advantages for production of carminic acid because the higher titer carminic acid would provide efficiency in purifying and/or concentrating carminic acid from the reaction mixture. In certain embodiments, the methods of the invention provide carminic acid titer value of from at least 5-fold higher than the conventional methods. In certain embodiments, the methods of the invention provide carminic acid titer value of from at least 10-fold higher than the conventional methods. In certain embodiments, the methods of the invention provide carminic acid titer value of from at least 50-fold higher than the conventional methods. In certain embodiments, the methods of the invention provide carminic acid titer value of from at least 100-fold higher than the conventional methods. In certain embodiments, the methods of the invention provide carminic acid titer value of from at least 500-fold higher than the conventional methods. In certain embodiments, the methods of the invention provide carminic acid titer value of from at least 1000-fold higher than the conventional methods.

In some embodiments, the isolated carminic acid has a purity of about 10%, or about 20%, or about 30%, or about 40%, or about 50%, or about 60%, or about 70%, or about 80%, or about 90%, or about 95%, or about 99%, or about 100%.

In other embodiments, the isolated carminic acid has a purity of from about 10% to 95%, or from about 10% to 90%, or from about 10% to 80% or from about 10% to 70%, or from about 10% to 60%, or from about 10% to 50%, or from about 10% to 40%, or from about 20% to 95%, or from about 20% to 90%, or from about 20% to 80% or from about 20% to 70%, or from about 20% to 60%, or from about 20% to 50%, or from about 20% to 40%, or from about 50% to 95%, or from about 50% to 90%, or from about 50% to 80% or from about 50% to 70%, or from about 50% to 60%.

### VII. EXAMPLES

### Example 1: Bioproduction of Carminic Acid

The reaction conditions for the reactions are provided in Table 4 below. The exemplary reaction conditions provided below are for the reactions provided for the conversion of kermesic acid to carminic acid and/or conversion of sucrose to UDP-glucose.

TABLE 4
Reaction conditions:

| Component | Working Range | |
|---|---|---|
| Buffer | pH 6-8, 5-100 mM | |
| UDP-glucose | 0.01-5 | mM |
| Magnesium chloride | 1-20 | mM |
| Kermesic acid | 0.1-50 | g/L |
| Sucrose | 50-600 | mM |
| Host microbe lysate | 5-40% (v/v) | |
| with enzymes | | |

| | |
|---|---|
| Time | 0.5 h-48 h |
| Temperature | 20° C.-40° C. |

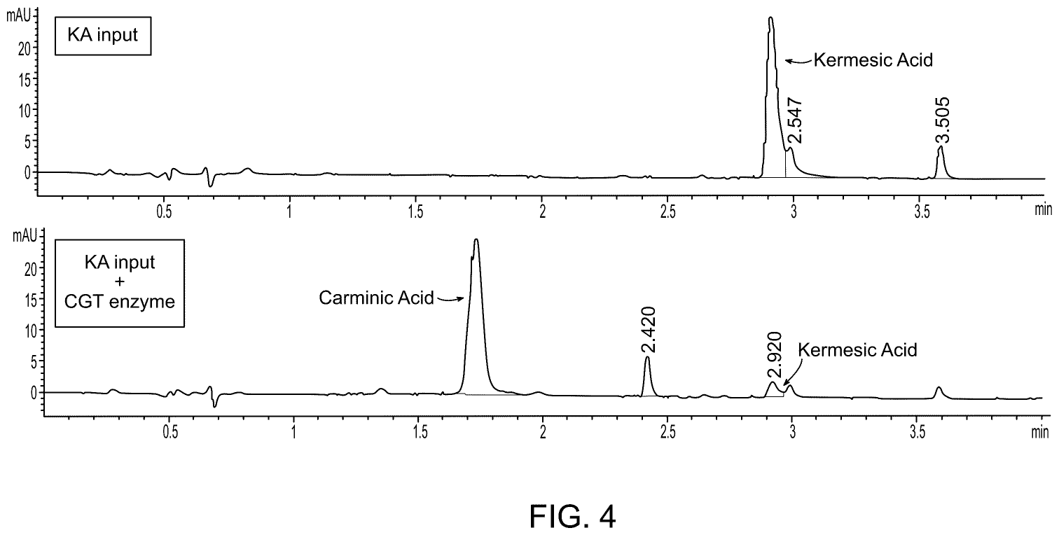

FIG. 4 provides the results from the reaction. As demonstrated in FIG. 4, upon addition of CGT to a mixture comprising kermesic acid, the kermesic acid is converted to carminic acid.

### Example 2: Reaction for Generation of UDP-Glucose and/or Recycling of Intermediates

The reaction conditions for the reactions for generation of UDP-glucose and/or recycling of intermediates are provided in Table 5 below.

TABLE 5
Reaction conditions

| Component | Working Range | |
|---|---|---|
| Buffer | pH 6-8, 5-200 mM | |
| Magnesium chloride | 1-100 | mM |
| Kermesic acid | 0.1-50 | g/L |
| Polyphosphate | 1-60 | g/L |
| UTP or UDP | 0.1-10 | μM |
| ATP | 0.1-10 | μM |
| Glucose, glucose-1-phosphate, | 0.01-500 | mM |
| glucose-6-phosphate, or UDP- | | |
| glucose | | |
| Host microbe lysate(s) with | 0.5-40% (v/v) | |
| enzymes | | |

| | |
|---|---|
| Time | 0.5 h-48 h |
| Temperature | 20° C.-40° C. |

In certain embodiments, the enzymes in the aforementioned Table 5 are CGT, SuSy, NDK, UGP, PPK, PGM, GLK and/or HK.

Table 6 provides exemplary sequences for glycosyltransferase enzymes in accordance with the methods of invention.

TABLE 6
Glycosyltransferase Enzymes

| SEQ | | | |
|---|---|---|---|
| ID | Accession # | Organism | Sequence |
| 1 | AXF38108 | Bergenia | MGSEPLVHVFLVSFPGQGHVNPLLRLGKRLAS |
| | | purpurascens | KGLLVTFSTPDSIGKQMRKASNLTDQPTPVGSG |
| | | | YIRFEFFEDGWAEDEPMRQDLDLYLPQLELVG |
| | | | KKLIPEMLKKHAEQDRPVSCLINNPFIPWVSDV |
| | | | ADELGIPSAMLWVQSTACFSAYYHYYHGIVPF |
| | | | PSETDPEIDVQLPHIPLLKYDEVPSFLHPTTPYPF |
| | | | LRRAILGQYKNLHKPFCILMETFYELEPEVIDY |
| | | | MSKLCPIKPVGPLFKNPTAPGTTVRGDFMTAD |
| | | | DCIEWLDSKPASSVVYISFGSVVYLKQEQIDEIA |
| | | | YGLLNAGISFLWVMKPPHKDAGLELLVLPDGF |
| | | | LEKAGDKGKMVQWSPQEQVLAHPSVACFVTH |
| | | | CGWNSTMESLTSGMPVVAFPQWGDQVTDAVY |
| | | | LCDVFKTGVRMCRGEAENRVIPRDEVEKCLLE |
| | | | AISGPKAEEMKKNALKWKELAEAAVAEGGSS |
| | | | DRNLQEFVDEVRRRSVGLTYKSTAGKIVEVVD |
| | | | SALPALAGQV |
| 2 | AFJ52990 | Linum | MGSSSSEKVLHVLLVCFPGQGHINPFLRLANLL |
| | | usitatissimum | ASHGLLVTFCINKTTGLKMKMSDNKSAVQFDF |
| | | | FDEGLDEEQIKVIPLDQLMNRLEETGRKALPEII |
| | | | EKHSENGQPVSCLVSNPFLPWVSDVAVSLDIPS |
| | | | AILWMQSCACESSYYHYHNKLARFPTENEPEC |
| | | | DVVLPSMPVLKHDEVPSFLHPSTPHPFLATAIL |
| | | | GQIAFLGKVFCILMETFQELEPEIIRHVSTLQNNI |
| | | | KPVGPLCLTGKISGGDLMEVDDDCIKWLDGKD |
| | | | ESSVVYISMGSIVSMDPTQREEFAYGLINSGLPF |
| | | | LWVVRPGHGESDGPGHQIIFPSVLEEKGKMVR |
| | | | WAPQEEVLRHPAVACFVTHCGWNSTMEAISA |
| | | | GKPVVTFPQWGDQVTDAKFLVDVFEVGVRMG |
| | | | RGATTTKMVKREEVERCVVEATVGEKAEMLR |
| | | | RNAARWKKEAEAAVAEDGSSTRSLLEFVEEVK |
| | | | KRNGTTS |
| 3 | KAG8368435 | Buddleja | MGSSVDHFDEGPLQNIHVFMVSFPGQGHVNPL |
| | | alternifolia | LRLGKRLASSGLFVTFSAPEFAGRSIRKSNKITE |
| | | | DEATPLGRGMIRFEFFDDGWALDHPGRDNLD |
| | | | MYLAQLERVGRETLPMMIRKQGEAGRPVSCLI |
| | | | NNPFIPWVSDVAEYLGMPSAVLWIQSCACFSA |
| | | | YYHFYHNLVPFPTENEPEIEVELPFMPSLKHDEI |
| | | | PSFLHPSTPYPFLGRAILGQFKNLHKPFCILMDT |
| | | | FQELENEIIEYTSKLIGRPIKTIGPLFKNSDDNSK |
| | | | SSDIRADFFAADDCIEWLDSKPERSVVYISFGSV |
| | | | VHLKQEQIDEIAYGLLNAGVSFLWVLRPPPKEH |
| | | | MNWVPHVLPEGFLGEAGDKGRIVGWAPQEAV |
| | | | LAHPSTACFVTHCGWNSTVEALTSGVPVVAFP |
| | | | QWGDQVTDAKFLVDVFRVGVRMCRGEAEDR |
| | | | VVGRDEVAMCLKAAVEGPEAAKMKENALKW |
| | | | KNAAAAAVAAGGSSYMNMQDFVEEVVRKSQ |
| | | | QKKM |
| 4 | AFJ52991 | Linum | MGSSSSEKALHVLLVCFPGQGHINPFLRLANLL |
| | | usitatissimum | ASHGLLVTFCINKTTGGQMKIPKNNLPSDNKPT |
| | | | IQFDFFDEGLDDEQIKVTPLDQLMTRLEETGRK |
| | | | ALPGIIEKYSENGQPVSCLVSNPFLPWVCDVAV |
| | | | SLDIPSAILWMQSCACFSSYYHYHNKLARFPTE |
| | | | NDAECDVVLPSMPVLKHDEVPSFLHPSTPYPFL |
| | | | ATAILGQFAYLDKVFCILMETFQELEPEIIRHVS |
| | | | TLHNNIKPVGPLCLTGKISGGDLMEVNDDCIK |
| | | | WLDGKDKSSVVYISMGSVVSMDPTQREEFAY |
| | | | GLMNSGLPFLWVVRPGYGEGDEPDHQIIFPSGL |
| | | | EGRGKMVRWAPQEEVLRHPAVACFVTHCGW |
| | | | NSTMEAISAGKPVVTFPQWGDQVTDAKFLVDV |
| | | | FEVGVRMGRGATTTKLVKRDEVERCVVEATV |
| | | | GEKAEVLRRNAMRWMKEAEAAVAEDGSSTRS |
| | | | LLEFVEEVKKRNGATL |
| 5 | AUI41123 | Rhodiola rosea | MGSEPLVHVFLVSFPGQGHVNPLLRLGKRLAS |
| | | | KGLLVTFTTPESIGHQMRKANKIVDGQPNPVG |
| | | | DGFLRFEFFEDGWDEHEVKRQDLDLYLPQLEL |
| | | | VGKVVIPQMIKRNAEAGRPVSCLINNPFIPWVS |
| | | | DVAESLGLPSAMLWVQSAACFSAYYHYSKNL |
| | | | VPFPSETDPEIDVQLPNMPLLKYDEVPSFLHPTT |
| | | | PYPFLRRAIMGQYNNLEKPFCVLMDTFDELEH |
| | | | DVIEFMSKFVPIRPVGPLFKNPKAPAAVRGDFM |
| | | | QADDCIGWLDAKPPGSVVYISFGSVVYLQQEQ |
| | | | VDEIAQGLLSSKVGFLWVMKPPHPDAGLELLK |
| | | | LPEGFLEEAGDRGKVVQWSPQEQVLAHPSVAA |
| | | | FVTHCGWNSTMEALTSGMPVIAFPQWGDQVT |
| | | | DAKYLVDEFKVGVRMCRGEAENRVVSREEVR |
| | | | KCLEEATVGEKAAEMKANAAKWKKAATEAFT |
| | | | EGGSSDRNLQAFVDDVRRKSLEIGKKSTKLGL |
| | | | DIDIPVAKDAISA |
| 6 | KAD6795309 | Mikania | METKHDLVHVFLVTFPAQGHVNPLLRLGKLIA |
| | | micrantha | SKGNVLVTFSATKSIGKRMKNAGAPVSGEPVP |
| | | | VGNRGGMLRFEFFDDGCSEHNDDERNDFVAY |
| | | | LPKLEAYGKTALTEIISHHAENGQPVSCLINNPF |
| | | | VPWVSDLAKDLNIPCAMLWVQSCSCFSAYYH |
| | | | YQNSLVTFPSEEQPDIDVQLPNMPVLKSDEIPSF |
| | | | LHPSTPYPFLRRAILGQFKNLSNNFCVLMETFEE |
| | | | LEGDLIKYMSQICQIRAVGPLFKNPMLDNSISG |
| | | | DLIKADDCLEWLDSKQPSSVVYISFGSIVSLNEE |
| | | | QVGEMAYGVLNSGVSFLWVMRTDAISTGEPG |
| | | | RLPVGFMEEAGERGMVVQWSPQAQVLAHPAV |
| | | | SCFVTHCGWNSTMEALSSGVPVVAFPQWGDQ |
| | | | VTDAKYLVDEWKVGIRMCRGEAEHRVIGRTE |
| | | | VEACIREATGGVNQAEMKMNAMKWKKAAEA |
| | | | AVAEGGSSDRNIQNFVDEIRNISFTKP |
| 7 | XP_006282251 | Capsella | MGFGSSSPSKPIHVMMVSFMGQGSVTPLFRLG |
| | | rubella | KLIASKGTVVTFVSPEFWAKKMREANKIVDSE |
| | | | LKPVGAGAIRFESYDKEWTEEDDSRGDFSAYM |
| | | | SHLEDAGKREVSKLVTSYMEKKDHVACLINNT |
| | | | FLPWVGDVAEEFNIPCALLWVQSAACFSAYYH |
| | | | YQDGSVPFPTETEPERDVKLPCFPVLKHDEIPNF |
| | | | LHPSSKYPGFSKAILGQFKNLRRPLCILMNSFNA |
| | | | LEQELIEYMSAFCQIKTVGPLSLFAKEVSSDVSA |
| | | | DMCKASDECIEWLDSRPKSSVVYISFGTVVYFK |
| | | | QEQMEEVIYGVLNSGLSFLWVIRPPVKAMRVE |
| | | | ANVLPQELFDAANSGRGKIVEWSPQEKVLAHP |
| | | | SVSCFVSHCGWNSTTEALSIGVPVVCLPKLGDQ |
| | | | VMNAVCLIDIFKMGVRLGRGAIVDRVAPRKEV |
| | | | TEKLLEAAVGEKAEELKKNALKWKAKAEAAV |
| | | | APGGSSDKSLWEFLEMIGVRVSQEKPNIHA |
| 8 | BAF18172 | Oryza sativa | TCGVSNTTNATTLVGFRGKHTTRAGGNKALRS |
| | | Japonica | MEPHVLLVSFPMQGHVNPLLRLGRRLAATGLL |
| | | Group | VTFTTVRLAAGGGRLRDVPEDGACADVGLGR |
| | | | LRFEYLRDDDDDGDERCQQLAPNDVLSHVTA |
| | | | VGPSALAEFIDGQADAGRPVTFVVNNIFVPWA |
| | | | LDVAAGMGIPCAMLWIQPCSVLSIYYHFYESPE |
| | | | AFPTAADPDVPVELPGLPVMAMVELPFMVRPE |
| | | | YAQCLWGDTLRAQVGAIKRTVSWVLVNSFYE |
| | | | LERSAVDALRAHTTVKLAPIGPLLEHGHDNGG |
| | | | GDDGAPAPALGAEDNDRCVAWLDAQPPRSVV |
| | | | YVAFGSLVNIGRDETAAVAEGLVATGRPFLWV |
| | | | VRDDSRDLVPEAVLAACRGDKAGKITAWCPQ |
| | | | GRVLAHGAVGCFVTHCGWNSIMEALAAGVPV |
| | | | VGYPWWSDQFANAKFLVEDYKVGVRLPAPVT |
| | | | GGELRACVDRVMSGPEAAVIRKRAMHWKREA |
| | | | AAAVADGGSSDRSLQDFVDHVRRSKGPEELAR |
| | | | LAQDIQIMNGPVNPVLV |
| 9 | A0A0B6VIJ5 | Gentiana | MGSLTNNDNLHIFLVCFIGQGVVNPMLRLGKA |
| | | trifloral | FASKGLLVTLSAPEIVGTEIRKANNLNDDQPIK |
| | | | VGSGMIRFEFFDDGWESVNGSKPFDVWVYINH |
| | | | LDQTGRQKLPIMLKKHEETGTPVSCLILNPLVP |
| | | | WVADVADSLQIPCATLWVQSCASFSAYYHYH |
| | | | HGLVPFPTESEPEIDVQLPGMPLLKYDEVPDYL |
| | | | HPRTPYPFFGTNILGQFKNLSKNFCILMDTFYEL |
| | | | EHEIIDNMCKLCPIKPIGPLFKIPKDPSSNGITGN |
| | | | FMKVDDCKEWLDSRPTSTVVYVSVGSVVYLK |
| | | | QEQVTEMAYGILNSEVSFLWVLRPPSKRIGTEP |
| | | | HVLPEEFWEKAGDRGKVVQWSPQEQVLAHPA |
| | | | TVGFLTHCGWNSTQEAISSGVPVITFPQFGDQV |
| | | | TNAKFLVEEFKVGVRLGRGELENRIITRDEVER |
| | | | ALREITSGPKAEEVKENALKWKKKAEETVAKG |
| | | | GYSERNLVGFIEEVARKTGTK |
| 10 | GKV43195.1 | Shorea | MGSESLVHVLLVSFPGQGHVNPLLRLGKRLAS |
| | | leprosula | KGLLVTFTTPETTGKQMRKASNVSEEPTAVGD |
| | | | GYIRFEFFEDGWDEDEPRRQDLDQYLPQLEKV |
| | | | GKIVIPEMIKRNAEQNRPVSCLINNPFIPWVSVV |
| | | | AESLGLPSAMLWVQSCACFAAYYHYHHGLVS |
| | | | FPSETDPEIDVQLPSMPLLKYDEVPSFLHPTTPY |
| | | | PFLRRAILGQYKSLDKPFCILMESFQELEPEIIEY |
| | | | MSKLCPIKPVGPLFKNPKAPNSAVRGDLMPQA |
| | | | DDCIDWLNTKPPSSVVYISFGSVVYLKQEQVDE |
| | | | IAHALLNSGITFLWVMKPPHKDSGYELLTLPEG |
| | | | FLEKVGNNGQVVQWSPQEKVLAHPAVACFVT |
| | | | HCGWNSTMESLSSGMPVVAFPQWGDQVTDAV |
| | | | YLTEVFKTGVRMCRGEAEDRIIARDEVEKCLK |
| | | | EAMVGPKAMEMKQNALKWKAAAEAAIAEGG |
| | | | SSDRNLQAFVDEVKRRSMEIAAGKSTKGVGGD |
| | | | LTKKSTENGKVEVEA |
| 11 | CAA7037192.1 | Microthlaspi | MDQNSFPLPPHVMLVSFPGQGHVNPLLRLGKL |
| | | erraticum | LASKGLLVTFVTTESWGKKMRTANNIQDRVLK |
| | | | PIGKGYIRFDFFDDGLPEDDDASRTDFTILRPQL |
| | | | ELVGQREIKSLVRRYEEVTKQPVTCLINNPFVS |
| | | | WVCDVAEDLQIPCAVLWVQSCACLASYYYYH |
| | | | HKLVDFPTKTDPKIDVQIPGMPLLKHDEIPSFIH |
| | | | PSSPYSALREVIIDQIKRLHKPFAVLIDTIHSLEQ |
| | | | DIIDHMSDVRLPGVIRPIGPLYKMAKTLASDEIN |
| | | | GDMSESTDHCMEWLDSQPVSSVVYISFGTVAY |
| | | | LKQEQINEIAYGVLNAGVSFLWVIRTQELGVN |
| | | | KERHVLPEEVKGKGKIVEWCSQEKVLAHPSIV |
| | | | CFVTHCGWNSTMEAVSSGVPTVCCPQWGDQV |
| | | | TDAVYMIDVLKTGVRLCRGETEERVVPREEVA |
| | | | ERLREITKGEKATELKKNALKWKEEAEAAVAR |
| | | | GGSSDRNIEEFVEKLGAKRVGKQNGGLEKONG |
| | | | GLAKONGSLNHVLS |
| 12 | CAD6239704.1 | Miscanthus | MSAREETAPAAAAAAPPHVVLVCFPSQGNLNP |
| | | lutarioriparius | TLRLAKRLAAKGLLVTCCTTSSIGACLAAASSS |
| | | | GVVSTGGDGVRVGSGRIRFEFLDDHGNEKDDL |
| | | | MRYFETSAPAAFVELLGRQAEAGRPVTYVVGN |
| | | | PFLPWVVDVAAEAGVPAAVLWVQSCAVLSLY |
| | | | YHYARGLVEFPPEDDIDARVALPGLPPLSVADV |
| | | | PSFLLPSNPYKMIADAIQGQFRNVDKAAWVFV |
| | | | NSFTELERDVLAALPGVTPRPPQLIPVGPLIELE |
| | | | EDGAAVRGDLIKAADDDCVGWLDAQPPRSVV |
| | | | YASVGSVVVLSAEEVAEMAHGLASTGRPFLW |
| | | | VVRPDTRPLLPEGFLDTVAGCGMVVPWSPQER |
| | | | VLEHAATACFLTHCGWNSTLETVAAGVPVVAF |
| | | | PQWGDOCTDAKFLVDELRMGVRLRAPLRREA |
| | | | VREAVDAAVAGPEADAMLSRARSWSAVARGA |
| | | | VAPGGSSDRHVQAFVDEVVQRACGRQPDEVT |
| | | | MMSG |
| 13 | XP_021750757 | Chenopodium | MGSETQIIHNNELDQLVHVFMISFPGQGHINPLL |
| | | quinoa | RLGKRIASKGFLVTFTTTENLGQGIRGSNDAVS |
| | | | DQPVPIGDGFIRFEFFDDEWPDGDPKKFDMDQ |
| | | | YLPQLEDVGRRWVPERLAVLAQEGRPVSCLIN |
| | | | NPFIPWVSDVAEELGLPSAMLWPQSCACFLAY |
| | | | YYFHHNMIPFPTEDALEIDVEVPTLPLLKWDEIP |
| | | | TFLHPTTPYAFLKRAILAQYKNLSKPFCILMDTF |
| | | | YELERSTVDHTIELLAPTPIRTIGPLFKKPIPGGA |
| | | | RVRADPLRPNQDCLKWLDGKPDGSVVYISFGT |
| | | | IVFPPQKQIDEIAAGIEAAGITFLWVMKPPAKEA |
| | | | KMSPHTLPDGFLDRIGDNGKVVQFAPQEQVLA |
| | | | HPAVSCFVTHCGWNSTMESLSYGVPMVAFPS |
| | | | WGDQVTDAKFICDVFKTGIQLTRGEHEKRLITK |
| | | | EEVEKCLRDATSGPKAVEMKKIALKWKDLAD |
| | | | EAIADGGSSDKNFDFFIDQVRKRGEIVVANAKK |
| | | | VANGLPTKENGH |
| 14 | BAG14302 | Gomphrena | MGSETHHSNDPQLTHIFMISFPGQGHINPLLRLG |
| | | globose | KRVASKGLLVTFATTENFGQYIRISNDAISDQP |
| | | | VPVGDGFIRLEFFDDEWPDGDPRKHDMDQYLP |
| | | | QLEKVGRKWVTQRLAALAHEYRPVSCLVNNP |
| | | | FLPWVSDLAEELGLCSAMLWPQSCACFLAYYY |
| | | | FHNNLVPFPSQDALEIDVEIPTLPLLKWDEIPTF |
| | | | LHPTTPYAFLKRAILAQYNNLTKPFCVLMDTFY |
| | | | ELEKPTVDHTIELLAPLPIKPVGPLFKKKVTGGS |
| | | | DVRADPIRPDQDCLSWLDGQPDGSVIYISFGTV |
| | | | VFLPQKQVDEIAAALEAADLSFLWVMKPPLKE |
| | | | SGWTPHCLPDGFLERVGQNGKVVQFAPQEQVL |
| | | | AHPALACFMTHCGWNSTMESLTSGVPVIAFPS |
| | | | WGDQVTDAKFLCDVYKTGIQLTRGEHEKKIIP |
| | | | RDEVEKCLREATSGPKAEEMKENALKWKAHA |
| | | | EETIADGGSSDQNIDFFVEGVRKRSEVVLAKAT |
| | | | NGVDANGYSLHIEANG |
| 15 | XP_031382117 | Punica | MGSESSLVHVFLVSFPGQGHVNPLLRLGKRLA |
| | | granatum | SKGLLVTFTTPESIGKQMRKASNISDQPAPVGD |
| | | | GFIRFEFFEDGWDEDEPRRQDLDQYLPQLEKV |
| | | | GKVLIPQMIQKNAEQGRPVSCLINNPFIPWVSD |
| | | | VAETLGLPSAMLWVQSCACFLAYYHYYHGLV |
| | | | PFPSENAMEIDVQLPSMPLLKHDEVPSFLYPTTP |
| | | | YPFLRRAILGQYKNLEKPFCILMDTFQELEHEII |
| | | | EYTSKICPIKTVGPLFKNPKAPNTTVKGDFMKA |
| | | | DDCIGWLDSKPASSVVYVSFGSVVYLKQDQW |
| | | | DEIAYGLLNSGVNFLWVMKPPHKDSGYTVLTL |
| | | | PEGFLEKAGDRGKVVQWSPQEQVLAHPATACF |
| | | | VTHCGWNSSMEALTSGMPVVAFPQWGDQVTD |
| | | | AKYLVDEFKVGVRMCRGEAEDKLITRDVVEQ |
| | | | CLREATQGPKAAEMKKNALKWKAAAEASFVE |
| | | | GGSSDRNLQAFVDEVKRRSIEITASKPAVKAAP |
| | | | NGVVAAAESVVETKANGKVELAA |

Table 7 provides exemplary sequences for sucrose synthase enzymes in accordance with the methods of invention.

TABLE 7
Sucrose Synthase Enzymes

| SEQ | | | |
|---|---|---|---|
| ID | Accession # | Organism | Sequence |
| 16 | WP_004872341.1 | Acidithiobacillus | MIEALRQQLLDDPRSWYAFLRHLVASQRDS |
| | | caldus | WLYTDLQRACADFREQLPEGYAEGIGPLEDF |
| | | | VAHTQEVIFRDPWMVFAWRPRPGRWIYVRIH |
| | | | REQLALEELSTDAYLQAKEGIVGLGAEGEAV |
| | | | LTVDFRDFRPVSRRLRDESTIGDGLTHLNRRL |
| | | | AGRIFSDLAAGRSQILEFLSLHRLDGQNLMLS |
| | | | NGNTDFDSLRQTVQYLGTLPRETPWAEIRED |
| | | | MRRRGFAPGWGNTAGRVRETMRLLMDLLDS |
| | | | PSPAALESFLDRIPMISRILIVSIHGWFAQDKVL |
| | | | GRPDTGGQVVYILDQARALEREMRNRLRQQ |
| | | | GVDVEPRILIATRLIPESDGTTCDQRLEPVVGA |
| | | | ENVQILRVPFRYPDGRIHPHWISRFKIWPWLE |
| | | | RYAQDLEREVLAELGSRPDLIIGNYSDGNLVA |
| | | | TLLSERLGVTQCNIAHALEKSKYLYSDLHWR |
| | | | DHEQDHHFACQFTADLIAMNAADIIVTSTYQ |
| | | | EIAGNDREIGQYEGHQDYTLPGLYRVENGID |
| | | | VFDSKFNIVSPGADPRFYFSYARTEERPSFLEP |
| | | | EIESLLFGREPGADRRGVLEDRQKPLLLSMAR |
| | | | MDRIKNLSGLAELYGRSSRLRGLANLVIIGGH |
| | | | VDVGNSRDAEEREEIRRMHEIMDHYQLDGQL |
| | | | RWVGALLDKTVAGELYRVVADGRGVFVQPA |
| | | | LFEAFGLTVIEAMSSGLPVFATRFGGPLEIIED |
| | | | GVSGFHIDPNDHEATAERLADFLEAARERPK |
| | | | YWLEISDAALARVAERYTWERYAERLMTIAR |
| | | | IFGFWRFVLDRESQVMERYLQMFRHLQWRPL |
| | | | AHAVPME |
| 17 | P30298.2 | Oryza sativa | MAAKLARLHSLRERLGATFSSHPNELIALFSR |
| | | Japonica Group | YVNQGKGMLQRHQLLAEFDALIEADKEKYA |
| | | | PFEDILRAAQEAIVLPPWVALAIRPRPGVWDY |
| | | | IRVNVSELAVEELSVSEYLAFKEQLVDGHTNS |
| | | | NFVLELDFEPFNASFPRPSMSKSIGNGVQFLN |
| | | | RHLSSKLFQDKESLYPLLNFLKAHNHKGTTM |
| | | | MLNDRIQSLRGLQSSLRKAEEYLMGIPQDTPY |
| | | | SEFNHRFQELGLEKGWGDCAKRVLDTIHLLL |
| | | | DLLEAPDPANLEKFLGTIPMMFNVVILSPHGY |
| | | | FAQSNVLGYPDTGGQVVYILDQVRALENEML |
| | | | LRIKQQGLDITPKILIVTRLLPDAVGTTCGQRV |
| | | | EKVIGTEHTDILRVPFRSENGILRKWISRFDV |
| | | | WPFLETYTEDVANEIMREMQAKPDLIIGNYS |
| | | | DGNLVATLLAHKLGVTQCTIAHALEKTKYPN |
| | | | SDIYLDKFDSQYHFSCQFTADLIAMNHTDFIIT |
| | | | STFQEIAGSKDTVGQYESHIAFTLPGLYRVVH |
| | | | GIDVFDPKFNIVSPGADMSVYFPYTEADKRLT |
| | | | AFHPEIEELLYSEVENDEHKFVLKDKNKPIIFS |
| | | | MARLDRVKNMTGLVEMYGKNAHLRDLANL |
| | | | VIVCGDHGNQSKDREEQAEFKKMYGLIDQY |
| | | | KLKGHIRWISAQMNRVRNGELYRYICDTKGV |
| | | | FVQPAFYEAFGLTVIEAMTCGLPTIATCHGGP |
| | | | AEIIVDGVSGLHIDPYHSDKAADILVNFFEKC |
| | | | KQDSTYWDNISQGGLQRIYEKYTWKLYSERL |
| | | | MTLTGVYGFWKYVSNLERRETRRYIEMFYAL |
| | | | KYRSLASAVPLAVDGESTSK |
| 18 | Q820M5.1 | Nitrosomonas | MTTIDTLATCTQQNRDAVYTLLRRYFTANRT |
| | | europaea ATCC | LLLQSDLREGLLQTEQDCGQSDMLRAFVFRL |
| | | 19718 | QEGIFSSPWAYLALRPEIAKWEFMRIHQEHLIP |
| | | | EKLTISEFLKFKETVVKGEATESVLEVDFGPF |
| | | | NRGFPRLKESRSIGQGVIFLNRKLSSEMFSRIE |
| | | | AGHTSLLHFLGVHAIEGQQLMFSNNSHDIHA |
| | | | VRNQLRQALEMLETLDGTTPWIELAPKMNQL |
| | | | GFAPGWGHNANRVAETMNMLMDILEAPSPS |
| | | | ALEEFLACIPMISRLLILSPHGYFGQDNVLGLP |
| | | | DTGGQVVYILDQVRALEKEMHDRLQLQGVQ |
| | | | VEPKILIVTRLIPDAGDTTCNQRLEKVSGCTNT |
| | | | WILRVPFRKHNGEIIPHWISRFEIWPHLEIFAG |
| | | | DVEREALAELGGHPDLIIGNYSDGNLVATLLS |
| | | | RRLGVTQCNIAHALEKTKYLHSDIYWQENED |
| | | | KYHFSCQYTADLLAMNSADFIVTSTYQEIAGT |
| | | | REAEGQYESYQAFSMPDLYRVIHGIDLFDPKF |
| | | | NIVSPGANADIYFPYSDPNRRLHSLIPEIESLIF |
| | | | DDATNLPARGYLQDPDKPLIFTMARLDRIKNI |
| | | | TGLVELYAASPRLRSLANLVIVGGKIDPQHSS |
| | | | DHEEQEQIHRMHQLMDEHELDQQVRWLGM |
| | | | RLDKNLAGELYRYIADKRGIFVQPALFEAFGL |
| | | | TIIEAMASGLPTFATRYGGPLEIIQNNRSGFHI |
| | | | DPNQGAATADLIADFFEKNLENPQEWERISQ |
| | | | GALDRVASRYTWKLYAERMMTLSRIYGFWK |
| | | | FVSGLEREETDRYLNMFYHLQFRPLANRLAH |
| | | | EI |
| 19 | P13708.2 | Glycine max | MATDRLTRVHSLRERLDETLTANRNEILALLS |
| | | | RIEAKGKGILQHHQVIAEFEEIPEENRQKLTDG |
| | | | AFGEVLRSTQEAIVLPPWVALAVRPRPGVWE |
| | | | YLRVNVHALVVEELQPAEYLHFKEELVDGSS |
| | | | NGNFVLELDFEPFNAAFPRPTLNKSIGNGVQF |
| | | | LNRHLSAKLFHDKESLHPLLEFLRLHSVKGKT |
| | | | LMLNDRIQNPDALQHVLRKAEEYLGTVPPET |
| | | | PYSEFEHKFQEIGLERGWGDNAERVLESIQLL |
| | | | LDLLEAPDPCTLETFLGRIPMVFNVVILSPHGY |
| | | | FAQDNVLGYPDTGGQVVYILDQVRALENEM |
| | | | LHRIKQQGLDIVPRILIITRLLPDAVGTTCGQR |
| | | | LEKVFGTEHSHILRVPFRTEKGIVRKWISRFEV |
| | | | WPYLETYTEDVAHELAKELQGKPDLIVGNYS |
| | | | DGNIVASLLAHKLGVTQCTIAHALEKTKYPES |
| | | | DIYWKKLEERYHFSCQFTADLFAMNHTDFIIT |
| | | | STFQEIAGSKDTVGQYESHTAFTLPGLYRVVH |
| | | | GIDVFDPKFNIVSPGADQTIYFPHTETSRRLTS |
| | | | FHPEIEELLYSSVENEEHICVLKDRSKPIIFTMA |
| | | | RLDRVKNITGLVEWYGKNAKLRELVNLVVV |
| | | | AGDRRKESKDLEEKAEMKKMYGLIETYKLN |
| | | | GQFRWISSQMNRVRNGELYRVICDTRGAFVQ |
| | | | PAVYEAFGLTVVEAMTCGLPTFATCNGGPAE |
| | | | IIVHGKSGFHIDPYHGDRAADLLVDFFEKCKL |
| | | | DPTHWDKISKAGLORIEEKYTWQIYSQRLLTL |
| | | | TGVYGFWKHVSNLDRRESRRYLEMFYALKY |
| | | | RKLAESVPLAAE |
| 20 | CAA09297.1 | Nostoc sp. PCC | MSELMQAILDSEEKHDLRGFISELRQQDKNY |
| | | 7119 | LLRNDILNVYAEYCSKCQKPETSYKESNLSKL |
| | | | IYYTQEIIPEDSNFCFIIRPKIAAQEVYRLTADL |
| | | | DVEPMTVQELLDLRDRLVNKFHPYEGDILEL |
| | | | DFGPFYDYTPTIRDPKNIGKGVQYLNRYLSSK |
| | | | LFQDSQQWLESLFNFLRLHNYNGIQLLINHQI |
| | | | QSQQQLSQQVKNALNFVSDRPNDEPYEQFRL |
| | | | QLQTMGFEPGWGNTASRVRDTLNILDELIDSP |
| | | | DPQTLEAFISRIPMIFRIVLVSAHGWFGQEGVL |
| | | | GRPDTGGQVVYVLDQAKNLEKQLQEDAILA |
| | | | GLEVLNVQPKVIILTRLIPNSDGTLCNQRLEK |
| | | | VYGTENAWILRVPLREFNPKMTQNWISRFEF |
| | | | WPYLETFAIDSERELLAEFQGRPDLIVGNYTD |
| | | | GNLVAFLLTRRMKVTQCNIAHALEKSKYLFS |
| | | | NLYWQDLEEKYHFSLOFTADLIAMNAANFVI |
| | | | SSTYQEIVGTPDSIGQYESYKCFTMPELYHVV |
| | | | NGIELFSPKFNVVPPGVNENSYFPYTQTQNRIE |
| | | | SDRDRLEEMLFTLEDSSQIFGKLDDPNKRPIFS |
| | | | MARLDRIKNLTGLAECFGQSQELQERCNLILV |
| | | | AGKLRIEESEDNEEKDEIVKLYRIIDEYNLHG |
| | | | KIRWLGVRLSKNDSGEIYRVICDRQGIFVQPA |
| | | | LFEAFGLTILESMISGLPTFATQFGGPLEIIQDK |
| | | | INGFYINPTHLEETATKILDFVTKCEQNPNYW |
| | | | NIISEKAIDRVYSTYTWKIHTTKLLTLARIYGF |
| | | | WNFTSKEKREDLLRYLESLFYLIYKPRAQQLL |
| | | | EQHKYR |
| 21 | P49036.1 | Zea mays | MGEGAGDRVLSRLHSVRERIGDSLSAHPNEL |
| | | | VAVFTRLKNLGKGMLQPHQIIAEYNNAIPEAE |
| | | | REKLKDGAFEDVLRAAQEAIVIPPWVALAIRP |
| | | | RPGVWEYVRVNVSELAVEELRVPEYLQFKEQ |
| | | | LVEEGPNNNFVLELDFEPFNASFPRPSLSKSIG |
| | | | NGVQFLNRHLSSKLFHDKESMYPLLNFLRAH |
| | | | NYKGMTMMLNDRIRSLSALQGALRKAEEHL |
| | | | STLQADTPYSEFHHRFQELGLEKGWGDCAKR |
| | | | AQETIHLLLDLLEAPDPSTLEKFLGTIPMVFNV |
| | | | VILSPHGYFAQANVLGYPDTGGQVVYILDQV |
| | | | RAMENEMLLRIKQCGLDITPKILIVTRLLPDAT |
| | | | GTTCGQRLEKVLGTEHCHILRVPFRTENGIVR |
| | | | KWISRFEVWPYLETYTDDVAHEIAGELQANP |
| | | | DLIIGNYSDGNLVACLLAHKMGVTHCTIAHA |
| | | | LEKTKYPNSDLYWKKFEDHYHFSCQFTTDLI |
| | | | AMNHADFIITSTFQEIAGNKDTVGQYESHMA |
| | | | FTMPGLYRVVHGIDVFDPKFNIVSPGADLSIY |
| | | | FPYTESHKRLTSLHPEIEELLYSQTENTEHKFV |
| | | | LNDRNKPIIFSMARLDRVKNLTGLVELYGRN |
| | | | KRLQELVNLVVVCGDHGNPSKDKEEQAEFK |
| | | | KMFDLIEQYNLNGHIRWISAQMNRVRNGELY |
| | | | RYICDTKGAFVQPAFYEAFGLTVVEAMTCGL |
| | | | PTFATAYGGPAEIIVHGVSGYHIDPYQGDKAS |
| | | | ALLVDFFDKCQAEPSHWSKISQGGLQRIEEKY |
| | | | TWKLYSERLMTLTGVYGFWKYVSNLERRET |
| | | | RRYLEMLYALKYRTMASTVPLAVEGEPSSK |
| 22 | P49040.3 | Arabidopsis | MANAERMITRVHSQRERLNETLVSERNEVLA |
| | | thaliana | LLSRVEAKGKGILQQNQIIAEFEALPEQTRKK |
| | | | LEGGPFFDLLKSTQEAIVLPPWVALAVRPRPG |
| | | | VWEYLRVNLHALVVEELQPAEFLHFKEELVD |
| | | | GVKNGNFTLELDFEPFNASIPRPTLHKYIGNG |
| | | | VDFLNRHLSAKLFHDKESLLPLLKFLRLHSHQ |
| | | | GKNLMLSEKIQNLNTLQHTLRKAEEYLAELK |
| | | | SETLYEEFEAKFEEIGLERGWGDNAERVLDMI |
| | | | RLLLDLLEAPDPCTLETFLGRVPMVFNVVILS |
| | | | PHGYFAQDNVLGYPDTGGQVVYILDQVRAL |
| | | | EIEMLQRIKQQGLNIKPRILILTRLLPDAVGTT |
| | | | CGERLERVYDSEYCDILRVPFRTEKGIVRKWI |
| | | | SRFEVWPYLETYTEDAAVELSKELNGKPDLII |
| | | | GNYSDGNLVASLLAHKLGVTQCTIAHALEKT |
| | | | KYPDSDIYWKKLDDKYHFSCQFTADIFAMNH |
| | | | TDFIITSTFQEIAGSKETVGQYESHTAFTLPGL |
| | | | YRVVHGIDVFDPKFNIVSPGADMSIYFPYTEE |
| | | | KRRLTKFHSEIEELLYSDVENKEHLCVLKDKK |
| | | | KPILFTMARLDRVKNLSGLVEWYGKNTRLRE |
| | | | LANLVVVGGDRRKESKDNEEKAEMKKMYD |
| | | | LIEEYKLNGQFRWISSQMDRVRNGELYRYIC |
| | | | DTKGAFVQPALYEAFGLTVVEAMTCGLPTFA |
| | | | TCKGGPAEIIVHGKSGFHIDPYHGDQAADTLA |
| | | | DFFTKCKEDPSHWDEISKGGLQRIEEKYTWQI |
| | | | YSQRLLTLTGVYGFWKHVSNLDRLEARRYLE |
| | | | MFYALKYRPLAQAVPLAQDD |
| 23 | Q9M111.1 | Arabidopsis | MANPKLTRVLSTRDRVQDTLSAHRNELVALL |
| | | thaliana | SRYVDQGKGILQPHNLIDELESVIGDDETKKS |
| | | | LSDGPFGEILKSAMEAIVVPPFVALAVRPRPG |
| | | | VWEYVRVNVFELSVEQLTVSEYLRFKEELVD |
| | | | GPNSDPFCLELDFEPFNANVPRPSRSSSIGNGV |
| | | | QFLNRHLSSVMFRNKDCLEPLLDFLRVHKYK |
| | | | GHPLMLNDRIQSISRLQIQLSKAEDHISKLSQE |
| | | | TPFSEFEYALQGMGFEKGWGDTAGRVLEMM |
| | | | HLLSDILQAPDPSSLEKFLGMVPMVFNVVILS |
| | | | PHGYFGQANVLGLPDTGGQVVYILDQVRALE |
| | | | TEMLLRIKRQGLDISPSILIVTRLIPDAKGTTCN |
| | | | QRLERVSGTEHTHILRVPFRSEKGILRKWISRF |
| | | | DVWPYLENYAQDAASEIVGELQGVPDFIIGN |
| | | | YSDGNLVASLMAHRMGVTQCTIAHALEKTK |
| | | | YPDSDIYWKDFDNKYHFSCQFTADLIAMNNA |
| | | | DFIITSTYQEIAGTKNTVGQYESHGAFTLPGLY |
| | | | RVVHGIDVFDPKFNIVSPGADMTIYFPYSEET |
| | | | RRLTALHGSIEEMLYSPDQTDEHVGTLSDRSK |
| | | | PILFSMARLDKVKNISGLVEMYSKNTKLRELV |
| | | | NLVVIAGNIDVNKSKDREEIVEIEKMHNLMK |
| | | | NYKLDGQFRWITAQTNRARNGELYRYIADTR |
| | | | GAFAQPAFYEAFGLTVVEAMTCGLPTFATCH |
| | | | GGPAEIIEHGLSGFHIDPYHPEQAGNIMADFFE |
| | | | RCKEDPNHWKKVSDAGLQRIYERYTWKIYSE |
| | | | RLMTLAGVYGFWKYVSKLERRETRRYLEMF |
| | | | YILKFRDLVKTVPSTADD |
| 24 | Q8W517 | Ipomoea batatas | MAGNDWINSYLEAILDVGPGIDDAKSSLLLRE |
| | | | RGRFSPTRYFVEEVITGFDETDLHRSWVRAQ |
| | | | ATRSPQERNTRLENMCWRIWNLARQKKQLE |
| | | | GEQAQRLAKRRQERERGRREAVADMSEDLS |
| | | | EGEKGDAISDISAHGESIKGRLPRISSVETMES |
| | | | WANQQKGKKLYIVLISLHGLIRGENMELGRD |
| | | | SDTGGQVKYVVELARALGSMPGVYRVDLLT |
| | | | RQVSSPEVDWSYGEPTEMLTPINSEGLMTEM |
| | | | GESSGAYIIRIPFGPRDKYIPKEDLWPYIPEFVD |
| | | | GALNHILHVSKVLGGQIGSGRDVWPVAIHGH |
| | | | YADAGDSAALLSGALNVPMLFTGHSLGRDK |
| | | | LEQLLRQGRLSKDEINSTYKIMRRIEAEELSLD |
| | | | ASEIVITSTRQEIDEQWRLYDGFDPILERKLRA |
| | | | RIKRNVSCYGRFMPRMVVIPPGMEFHHIVPHE |
| | | | GDMDFETEGSEDGKAPDPHIWTEIMRFFSNPR |
| | | | KPMILALARPDPKKNLTTLVKAFGECRPLREL |
| | | | ANLTLIMGNRDNIDEMSSTNASVLLSILKMID |
| | | | KYDLYGQVAYPKHHKQSEVPDIYRLAAKTK |
| | | | GVFINPAFIEPFGLTLIEAAAHGLPIVATKNGG |
| | | | PVDIHRGSDNGLLVDPHDQHAIADALLKLVA |
| | | | DKHLWAKCRANGLKNIHLFSWPEHCKTYLS |
| | | | RIAGCKPRQPCWLRNADDDENSESESPSDSLR |
| | | | DIQDISLNLKFSLDGDKNEDSDNLFDPDDRKN |
| | | | KLENAVLAWSKGVKGTHKTSIDKIDQSSSAG |
| | | | KFPALRRRKQIFVIAVDCDSSTGLFENVRKIFA |
| | | | AVEAEGMEGSIGFHIGHFIQYIRSAFFSDFRGH |
| | | | ESTDFDAFICNSGGDLYYSSSHSEDNPFVVDL |
| | | | YYHSHIEYRWGGEGLRKTLVRWAASISDKKG |
| | | | EKEEHIVVEDEKNSADYCYTFKVQKSGGDPS |
| | | | VKELRKSMRIQALRCHVVYCQNGSRINVIPVL |
| | | | SSRSQALRYLYLRWGMDLSKLVVFVGESGDT |
| | | | DYEGLLGGLRKAVILKGVCSVSSSQLLSNRN |
| | | | YPLTDVVPYNSPNVIQTTEECSSSELHASLEKL |
| | | | AVLKG |
| 25 | WP_011056890 | Thermosynechococcus | MTCVLLKAVVESDERADLRQFSRILQLGEKR |
| | | elongatus | YLLRNDILDAFADYCRDQERPVPPPSESRLSK |
| | | | LVFYTQEIIVDNESLCWIVRPRIAQQEVCRLLV |
| | | | EDLTIVPMTIPELLDLRDRLVNHYHPNEGDVF |
| | | | EIDVQPFYDYSPIIRDAKNIGKGVEFLNRYLSS |
| | | | KLFQDPRQWQQNLFNFLRIHRYNGYQLLINE |
| | | | RIRSPQHLSEQVKQALVVLSDRPPTEAYSEFR |
| | | | FELQNLGFEPGWGNTVARVRDTLEILDQLLD |
| | | | SPDHQVLEAFVSRIPMLFRIALISPHGWFGQE |
| | | | GVLGRPDTGGQVVYILDQVKSLEKQMREDLE |
| | | | LAGLGVLEAQPKIIVLTRLIPNAEGTLCNQRLE |
| | | | KIYGTNDAWILRVPFREFNPKVTQNWISRFEI |
| | | | WPYLETFAIDAERELRAEFGHVPDLIIGNYSD |
| | | | GNLVAFLLARRLKVTQCNIAHALEKSKYLFS |
| | | | NLYWQDLEDKYHFSLOFTADLIAMNAANFIIS |
| | | | STYQEIVGTPDSIGQYESYQSFTMPDLYHVVN |
| | | | GIELFSPKFNVVPPGVNEQVYFPYYHYTERLE |
| | | | GDRQRLEELLFTLEDPQQIYGYLEAPEKRPLF |
| | | | SMARLDRIKNLTGLAEAFGRSKALQERCNLIL |
| | | | VAGKLRTADSSDREEIAEIEKLYQIIHQYNLH |
| | | | GKIRWLGIRLPKADSGEIYRIIADRQGIFVQPA |
| | | | LFEAFGLTILEAMISGLPTFGTRFGGPLEIIQDG |
| | | | VNGFYINPTHLEEMAETIVRFLEACDRDPQE |
| | | | WQRISKAGIERVYSTYTWKIHCTRLLSLAKIY |
| | | | GFWNFSSQENREDMMRYMEALFHLLYKPRA |
| | | | QALLAEHLQR |

Table 8 provides exemplary sequences for enzymes involved in generation and/or recycling of UDP-glucose the reaction intermediates in accordance with the methods of invention.

TABLE 8

| SEQ | | | |
|---|---|---|---|
| ID | | | |
| NO | Gene | Accession | Sequence |
| 26 | GLK | AAA27694.1 | MEIVAIDIGGTHARFSIAEVSNGRVLSLGEETTFKTAEHASLQ |
| | | | LAWERFGEKLGRPLPRAAAIAWAGPVHGEVLKLTNNPWVL |
| | | | RPATLNEKLDIDTHVLINDFGAVAHAVAHMDSSYLDHICGP |
| | | | DEALPSDGVITILGPGTGLGVAHLLRTEGRYFVIETEGGHIDF |
| | | | APLDRLEDKILARLRERFRRVSIERIISGPGLGNIYEALAAIEG |
| | | | VPFSLLDDIKLWQMALEGKDNLAEAALDRFCLSLGAIAGDL |
| | | | ALAQGRTSVVIGGGVGLRIASHLPESGFRQRFVSKGRFERV |
| | | | MSKIPVKLITYPQPGLLGAQLPMPTNILKLNNIF |
| 27 | GLK | WP_331012003.1 | MEIVAVDIGGTHARFAIAEVGDGHVLSLGEPVTLKTAEHGS |
| | | | LQLAWEAAGEALGRPIPRAAGIAIATPIGGEVLKLTNNPWVI |
| | | | RPALIQSKLGVDSYVLVNDFEAVGHAVAQVGAEYLQHLCG |
| | | | PDDPLPDTGIITIVGPGTGLGVAHLLKTPAGYHVLPCEGGHI |
| | | | DFAPLDVLEDGILKTLRKQYRRVSVERIVSGPGLVNIYESLPP |
| | | | STSAALRPIDDKELWTAALAGTDSRAAAALDRFCLSLGAVA |
| | | | GDLALAHGAKAVVIAGGLGLRIADHLPRSGFSERFVAKGRF |
| | | | ERIMSAMPVKFITHPQPGLFGAAAAFAGAHRS |
| 28 | PGM | AAC73782.1 | MAIHNRAGQPAQQSDLINVAQLTAQYYVLKPEAGNAEHAV |
| | | | KFGTSGHRGSAARHSFNEPHILAIAQAIAEERAKNGITGPCY |
| | | | VGKDTHALSEPAFISVLEVLAANGVDVIVQENNGFTPTPAVS |
| | | | NAILVHNKKGGPLADGIVITPSHNPPEDGGIKYNPPNGGPAD |
| | | | TNVTKVVEDRANALLADGLKGVKRISLDEAMASGHVKEQD |
| | | | LVQPFVEGLADIVDMAAIQKAGLTLGVDPLGGSGIEYWKRI |
| | | | GEYYNLNLTIVNDQVDQTFRFMHLDKDGAIRMDCSSECAM |
| | | | AGLLALRDKFDLAFANDPDYDRHGIVTPAGLMNPNHYLAV |
| | | | AINYLFQHRPQWGKDVAVGKTLVSSAMIDRVVNDLGRKLV |
| | | | EVPVGFKWFVDGLFDGSFGFGGEESAGASFLRFDGTPWSTD |
| | | | KDGIIMCLLAAEITAVTGKNPQEHYNELAKRFGAPSYNRLQ |
| | | | AAATSAQKAALSKLSPEMVSASTLAGDPITARLTAAPGNGA |
| | | | SIGGLKVMTDNGWFAARPSGTEDAYKIYCESFLGEEHRKQI |
| | | | EKEAVEIVSEVLKNA |
| 29 | PGM | A0A110EI47 | MAKHPRAGQQPTSCDLINVAKLTSQYYVLQPALHESAHAV |
| | | | KFGTSGHRGSSTKHTFNEAHILAIAQAIAEIRHAKGITGPCYV |
| | | | GKDTHALSEPAFISVLEVLAANQVTVIIQPENGFTPTPAISHAI |
| | | | LTHNKSNPIHLADGIVITPSHNPPEDGGIKYNPPNGGPADTDF |
| | | | TGLIEKRANQLLDERSSENAVLAGVKRISLSQAISSQFIIQQD |
| | | | MMMPYVEDLVNVVDLSVIQQAGLKLGVDPLGGSGIAYWQ |
| | | | QIAEYYKLNLSLVNDQVDQTFRFMPLDHDGVIRMDCSSVD |
| | | | AMGGLLRLKDKFDLAFANDPDYDRHGIVTPAGLMNPNHYL |
| | | | SVAINYLYQNRPGWPVDLGVGKTLVSSAMIDRVVSSLNRKL |
| | | | VEVPVGFKWFVDGLYTGKLGFGGEESAGASFLRFDAKPWS |
| | | | TDKDGIIMCLLSAEITARTGKNPQVHYDELSEKFGAPIYNRI |
| | | | QANATHEQKAILSKLSPDQVQAKTLAGDLITARITHAPSNN |
| | | | ASIGGLKVVTDNGWFAARPSGTEEAYKIYCESFISHEHLDLI |
| | | | SKEAVEIVDAAFASNK |
| 30 | PGM | A0A7M2S2U2 | MAMHPRAGQKAQQEDLHNIPALVANYFLLQPDAANAEHK |
| | | | VQFGTSGHRGTADKHTFNENHILAIAQAVAEVRSEQGTTGP |
| | | | LFVGKDTHALSEPAFSSVIEVLIANGVKVIVQQDNGYTPTPG |
| | | | ISHAILTYNIKHDEKADGIVITPSHNPPQDGGIKYNPTHGGPA |
| | | | EAELTQAIEDRANELIAEGLQGVKRLPLAEAKASDLFVEMD |
| | | | LVKPYIDDLVNVIDMEAIQKANLKIGVDPLGGSGIDYWRQI |
| | | | GQAYNLDLTLVSEAIDPSFQFMSLDKDGVVRMDCSSPYAM |
| | | | AGLLALKDEYDLAFGNDPDYDRHGIVTPKGLMNPNHFLAV |
| | | | CIDYLYRHRDAWGKDVAVGKTLVSSALIDRVVADLGRALC |
| | | | EVPVGFKWFVDGLYTGKFGFGGEESAGASFLRKDGTPWST |
| | | | DKDGILLCLLAAEITAVTGKNPQDYYEELAAKHGESKYNRI |
| | | | QAVANGPQKDVLKKLSPEMVAAETLAGDAITARLTHAPGN |
| | | | GAAIGGLKVTTANGWFAARPSGTEDIYKIYCESFKGEEHLK |
| | | | QIEAEAQEIVNQVFAAAGL |
| 31 | UGP | EEW2752841.1 | MAAINTKVKKAVIPVAGLGTRMLPATKAIPKEMLPLVDKPL |
| | | | IQYVVNECIAAGITEIVLVTHSSKNSIENHFDTSFELEAMLEK |
| | | | RVKRQLLDEVQSICPPHVTIMQVRQGLAKGLGHAVLCAHP |
| | | | VVGDEPVAVILPDVILDEYESDLSQDNLAEMIRRFDETGHSQ |
| | | | IMVEPVADVTAYGVVDCKGVELAPGESVPMVGVVEKPKA |
| | | | DVAPSNLAIVGRYVLSADIWPLLAKTPPGAGDEIQLTDAIDM |
| | | | LIEKETVEAYHMKGKSHDCGNKLGYMQAFVEYGIRHNTLG |
| | | | TEFKAWLEEEMGIKK |
| 32 | UGP | WP_021648042.1 | MFAEDLKRTEKMTVDDVFEQSAQKMREQGMSEIAISQFRH |
| | | | AYHVWASEKESAWIREDTVEPLHGVRSFHDVYKTIDHDKA |
| | | | VHAFAKTAFLKLNGGLGTSMGLQCAKSLLPVRRHKARQMR |
| | | | FLDIILGQVLTARTRLNVPLPVTFMNSFRTSDDTMKALRHQR |
| | | | KFKQTDIPLEIIQHQEPKIDAATGAPASWPANPDLEWCPPGH |
| | | | GDLFSTLRESGLLDTLLEHGFEYLFISNSDNLGARPSRTLAQ |
| | | | YFEDTGAPFMVEVANRTYADRKGGHIVRDTATGRLILREMS |
| | | | QVHPDDKDAAQDIAKHPYFNTNNIWVRIDVLRDMLAEHDG |
| | | | VLPLPVIINNKTVDPIDPQSPAVVQLETAMGAAIGLFEGAICV |
| | | | QVDRMRFLPVKTTNDLFIMRSDRFHLTDSYEMEDGNYIFPN |
| | | | VDLDPRYYKNIEDFNERFPYNVPSLAAANSVSIKGDWTFGR |
| | | | DVIMFADARLEDRNEPSYVPNGEYVGPMGIEPGDWV |
| 33 | UGP | WP_028847555.1 | MTASAEHQTFTTAIVPAAGLGTRFLPTTKSVPKELLPVVDTP |
| | | | AIELVADEARQAGAERLVIVTSPAKQSIAAYFRPAPELERSL |
| | | | EEKGKTGQLAKIRRAPELLEVEVAIQEQALGLGHAVACAEP |
| | | | NLGPEDDVVAVLLPDDLVLPHGILERMAKVRAEHGGSVLC |
| | | | AFDIPKEEISAYGVFDVSDTDDADVKRVHGMVEKPPAEQAP |
| | | | STFAAAGRYLLDRAIFDALRRIEPGAGGELQLTDAVALLIQE |
| | | | GHPVHVVVHRGDRHDLGNPGGFLRAAVDFALQDPDYGPEL |
| | | | RAWLTDRIARP |
| 34 | NDK | WP_000963837.1 | MAIERTFSIIKPNAVAKNVIGNIFARFEAAGFKIVGTKMLHLT |
| | | | VEQARGFYAEHDGKPFFDGLVEFMTSGPIVVSVLEGENAVQ |
| | | | RHRDLLGATNPANALAGTLRADYADSLTENGTHGSDSVES |
| | | | AAREIAYFFGEGEVCPRTR |
| 35 | NDK | CAF21035.1 | MTERTLILIKPDGVTNGHVGEIIARIERKGLKLAALDLRVAD |
| | | | RETAEKHYEEHADKPFFGELVEFITSAPLIAGIVEGERAIDA |
| | | | WRQLAGGTDPVAKATPGTIRGDFALTVGENVVHGSDSPES |
| | | | AEREISIWFPNL |
| 36 | NDK | WP_066795798 | MTERTLILIKPDGVANGHVGEIIARIERKGLKLVELDLRTAD |
| | | | RETAEKHYEEHSDKPFFGELVEFITSAPLVAGIVEGERAIDA |
| | | | WRQLAGGTDPVSKATPGTIRGDFALTVGENVVHGSDSPESA |
| | | | EREIAIWFPNK |
| 37 | PPK2 | WP_010968631 | MALDEAPAEARPGSRAVELEIDGRSRIFDIDDPDLPKWIDEE |
| | | | AFRSDDYPYKKKLDREEYEETLTKLQIELVKVQFWMQATG |
| | | | KRVMAVFEGRDAAGKGGAIHATTANMNPRSARVVALTKP |
| | | | TETERGQWYFQRYVATFPTAGEFVLFDRSWYNRAGVEPVM |
| | | | GFCTPDQYEQFLKEAPRFEEMIANEGIHLFKFWINIGREMQL |
| | | | KRFHDRRHDPLKIWKLSPMDIAALSKWDDYTGKRDRMLKE |
| | | | THTEHGPWAVIRGNDKRRSRINVIRHMLTKLDYDGKDEAAI |
| | | | GEVDEKILGSGPGFLR |
| 38 | PPK2 | WP_160857702 | MVEKAESRAVELDIDGKKRVFDIDDPKLPDWIDKEAFQSDD |
| | | | FPYDKKLDEDEYEEALEALQVELVKVQFWQQKTGARIMAI |
| | | | FEGRDAAGKGGAIHATMSNMNPRSARIVALTKPTETEQGQ |
| | | | WYFQRYVATFPTSGEFVLFDRSWYNRAGVEPVMGFCTPEQ |
| | | | YEKFLKATPRIEKMIASEGIHFFKFWLNIGREMQLKRFHDRR |
| | | | HDPLKVWKLSPMDIAALNKWDDYTEKRDRMLKATHTDRA |
| | | | PWTVIRANDKRRARINLIRHILTTLDYEGKDEKAIGKIDDKIV |
| | | | GSGPGFVK |

### INCORPORATION BY REFERENCE

References and citations to other documents, such as patents, patent applications, patent publications, journals, books, papers, web contents, publicly accessible databases, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

1. A method for cell-free production of carminic acid, wherein the method comprises: providing one or more enzymes in a cell-free medium, wherein the one or more enzymes result in transformation of one or more substrates to carminic acid.

2. The method of claim 1, wherein the substrate is kermesic acid.

3. The method of claim 2, wherein the enzyme is C-glucosyltransferase (CGT).

4. The method of claim 2, further comprising the cell-free medium comprises an activated sugar.

5. The method of claim 4, wherein the activated sugar is UDP-glucose.

6. The method of claim 5, wherein the UDP-glucose is added to the cell-free medium.

7. The method of claim 5, wherein the UDP-glucose is synthesized in the cell-free medium by the one or more enzymes.

8. The method of claim 7, wherein the UDP-glucose is synthesized from one or ingredients selected from the group consisting of: sucrose, glucose, UTP, UDP, ATP, glucose-6-phosphate, glucose-1-phosphate, and/or polyphosphate.

9. The method of claim 8, wherein the one or more enzymes is selected from the group consisting of sucrose synthase (SuSy), glucokinase (GLK), hexokinase (HK), phosphoglucomutase (PGM), polyphosphate kinase (PPK), UTP-glucose-1-phosphate uridylyltransferase (UGP), and nucleoside diphosphate kinase (NDK).

10. The method of claim 1, wherein the cell-free medium is a cell lysate.

11. The method of claim 10, wherein the cell lysate is a cell lysate from cells from a host organism expressing the one or more enzymes.

12. The method of claim 11, wherein the host organism is selected from a group consisting of: bacteria, yeast, and/or mammalian cells.

13. The method of claim 11, wherein the one or more enzymes are introduced in the host organism by integration into genome of the host organism or on a plasmid.

14. The method of claim 13, wherein the host organisms expressing the one or more enzymes are cultured until a pre-determined biomass is achieved to produce the requisite quantity of the one or more enzymes.

15. The method of claim 14, further comprising lysing of cells followed by removal of cell debris to generate a cell lysate for use in the cell-free medium for cell-free production of carminic acid.

16. The method of claim 15, wherein the one or enzymes are selected from the group consisting of CGT, SuSy, GLK, HK, PGM, PPK, UGP, and NDK.

17. The method of claim 16, wherein the one or more enzymes are present in the cell-free medium for cell-free production of carminic acid.

18. The method of claim 16, wherein the method does not include separating and/or purifying one more enzymes for cell-free production of carminic acid.

19. The method of claim 18, wherein the cell-free medium further comprises: buffer, kermesic acid, an activated sugar, magnesium chloride, cell lysate, sucrose, glucose, glucose-1-phosphate, glucose-6-phosphate, UDP, UTP, ATP, polyphosphate, and/or water.

20. (canceled)

21. (canceled)

22. The method of claim 18, wherein the method results in titer value of produced carminic acid is from about 10 times to about 5000 times higher than methods for cell-based production of carminic acid.

23.-74. (canceled)


SEQUENCE LISTING (38 sequences)

SEQ ID NO: 1 - protein, 495 aa, synthetic construct
MGSEPLVHVFLVSFPGQGHVNPLLRLGKRLASKGLLVTFSTPDSIGKQMRKASNLTDQPTPVGSGYIRFEFFEDGWAEDEPMRQDLDLYLPQLELVGKKLIPEMLKKHAEQDRPVSCLINNPFIPWVSDVADELGIPSAMLWVQSTACFSAYYHYYHGIVPFPSETDPEIDVQLPHIPLLKYDEVPSFLHPTTPYPFLRRAILGQYKNLHKPFCILMETFYELEPEVIDYMSKLCPIKPVGPLFKNPTAPGTTVRGDFMTADDCIEWLDSKPASSVVYISFGSVVYLKQEQIDEIAYGLLNAGISFLWVMKPPHKDAGLELLVLPDGFLEKAGDKGKMVQWSPQEQVLAHPSVACFVTHCGWNSTMESLTSGMPVVAFPQWGDQVTDAVYLCDVFKTGVRMCRGEAENRVIPRDEVEKCLLEAISGPKAEEMKKNALKWKELAEAAVAEGGSSDRNLQEFVDEVRRRSVGLTYKSTAGKIVEVVDSALPALAGQV

SEQ ID NO: 2 - protein, 462 aa, synthetic construct
MGSSSSEKVLHVLLVCFPGQGHINPFLRLANLLASHGLLVTFCINKTTGLKMKMSDNKSAVQFDFFDEGLDEEQIKVIPLDQLMNRLEETGRKALPEIIEKHSENGQPVSCLVSNPFLPWVSDVAVSLDIPSAILWMQSCACFSSYYHYHNKLARFPTENEPECDVVLPSMPVLKHDEVPSFLHPSTPHPFLATAILGQIAFLGKVFCILMETFQELEPEIIRHVSTLQNNIKPVGPLCLTGKISGGDLMEVDDDCIKWLDGKDESSVVYISMGSIVSMDPTQREEFAYGLINSGLPFLWVVRPGHGESDGPGHQIIFPSVLEEKGKMVRWAPQEEVLRHPAVACFVTHCGWNSTMEAISAGKPVVTFPQWGDQVTDAKFLVDVFEVGVRMGRGATTTKMVKREEVERCVVEATVGEKAEMLRRNAARWKKEAEAAVAEDGSSTRSLLEFVEEVKKRNGTTS

SEQ ID NO: 3 - protein, 488 aa, synthetic construct
MGSSVDHFDEGPLQNIHVFMVSFPGQGHVNPLLRLGKRLASSGLFVTFSAPEFAGRSIRKSNKITEDEATPLGRGMIRFEFFDDGWALDHPGRDNLDMYLAQLERVGRETLPMMIRKQGEAGRPVSCLINNPFIPWVSDVAEYLGMPSAVLWIQSCACFSAYYHFYHNLVPFPTENEPEIEVELPFMPSLKHDEIPSFLHPSTPYPFLGRAILGQFKNLHKPFCILMDTFQELENEIIEYTSKLIGRPIKTIGPLFKNSDDNSKSSDIRADFFAADDCIEWLDSKPERSVVYISFGSVVHLKQEQIDEIAYGLLNAGVSFLWVLRPPPKEHMNWVPHVLPEGFLGEAGDKGRIVGWAPQEAVLAHPSTACFVTHCGWNSTVEALTSGVPVVAFPQWGDQVTDAKFLVDVFRVGVRMCRGEAEDRVVGRDEVAMCLKAAVEGPEAAKMKENALKWKNAAAAAVAAGGSSYMNMQDFVEEVVRKSQQKKM

SEQ ID NO: 4 - protein, 468 aa, synthetic construct
MGSSSSEKALHVLLVCFPGQGHINPFLRLANLLASHGLLVTFCINKTTGGQMKIPKNNLPSDNKPTIQFDFFDEGLDDEQIKVTPLDQLMTRLEETGRKALPGIIEKYSENGQPVSCLVSNPFLPWVCDVAVSLDIPSAILWMQSCACFSSYYHYHNKLARFPTENDAECDVVLPSMPVLKHDEVPSFLHPSTPYPFLATAILGQFAYLDKVFCILMETFQELEPEIIRHVSTLHNNIKPVGPLCLTGKISGGDLMEVNDDCIKWLDGKDKSSVVYISMGSVVSMDPTQREEFAYGLMNSGLPFLWVVRPGYGEGDEPDHQIIFPSGLEGRGKMVRWAPQEEVLRHPAVACFVTHCGWNSTMEAISAGKPVVTFPQWGDQVTDAKFLVDVFEVGVRMGRGATTTKLVKRDEVERCVVEATVGEKAEVLRRNAMRWMKEAEAAVAEDGSSTRSLLEFVEEVKKRNGATL

SEQ ID NO: 5 - protein, 493 aa, synthetic construct
MGSEPLVHVFLVSFPGQGHVNPLLRLGKRLASKGLLVTFTTPESIGHQMRKANKIVDGQPNPVGDGFLRFEFFEDGWDEHEVKRQDLDLYLPQLELVGKVVIPQMIKRNAEAGRPVSCLINNPFIPWVSDVAESLGLPSAMLWVQSAACFSAYYHYSKNLVPFPSETDPEIDVQLPNMPLLKYDEVPSFLHPTTPYPFLRRAIMGQYNNLEKPFCVLMDTFDELEHDVIEFMSKFVPIRPVGPLFKNPKAPAAVRGDFMQADDCIGWLDAKPPGSVVYISFGSVVYLQQEQVDEIAQGLLSSKVGFLWVMKPPHPDAGLELLKLPEGFLEEAGDRGKVVQWSPQEQVLAHPSVAAFVTHCGWNSTMEALTSGMPVIAFPQWGDQVTDAKYLVDEFKVGVRMCRGEAENRVVSREEVRKCLEEATVGEKAAEMKANAAKWKKAATEAFTEGGSSDRNLQAFVDDVRRKSLEIGKKSTKLGLDIDIPVAKDAISA

SEQ ID NO: 6 - protein, 476 aa, synthetic construct
METKHDLVHVFLVTFPAQGHVNPLLRLGKLIASKGNVLVTFSATKSIGKRMKNAGAPVSGEPVPVGNRGGMLRFEFFDDGCSEHNDDERNDFVAYLPKLEAYGKTALTEIISHHAENGQPVSCLINNPFVPWVSDLAKDLNIPCAMLWVQSCSCFSAYYHYQNSLVTFPSEEQPDIDVQLPNMPVLKSDEIPSFLHPSTPYPFLRRAILGQFKNLSNNFCVLMETFEELEGDLIKYMSQICQIRAVGPLFKNPMLDNSISGDLIKADDCLEWLDSKQPSSVVYISFGSIVSLNEEQVGEMAYGVLNSGVSFLWVMRTDAISTGEPGRLPVGFMEEAGERGMVVQWSPQAQVLAHPAVSCFVTHCGWNSTMEALSSGVPVVAFPQWGDQVTDAKYLVDEWKVGIRMCRGEAEHRVIGRTEVEACIREATGGVNQAEMKMNAMKWKKAAEAAVAEGGSSDRNIQNFVDEIRNISFTKP

SEQ ID NO: 7 - protein, 485 aa, synthetic construct
MGFGSSSPSKPIHVMMVSFMGQGSVTPLFRLGKLIASKGTVVTFVSPEFWAKKMREANKIVDSELKPVGAGAIRFESYDKEWTEEDDSRGDFSAYMSHLEDAGKREVSKLVTSYMEKKDHVACLINNTFLPWVGDVAEEFNIPCALLWVQSAACFSAYYHYQDGSVPFPTETEPERDVKLPCFPVLKHDEIPNFLHPSSKYPGFSKAILGQFKNLRRPLCILMNSFNALEQELIEYMSAFCQIKTVGPLSLFAKEVSSDVSADMCKASDECIEWLDSRPKSSVVYISFGTVVYFKQEQMEEVIYGVLNSGLSFLWVIRPPVKAMRVEANVLPQELFDAANSGRGKIVEWSPQEKVLAHPSVSCFVSHCGWNSTTEALSIGVPVVCLPKLGDQVMNAVCLIDIFKMGVRLGRGAIVDRVAPRKEVTEKLLEAAVGEKAEELKKNALKWKAKAEAAVAPGGSSDKSLWEFLEMIGVRVSQEKPNIHA

SEQ ID NO: 8 - protein, 522 aa, synthetic construct
TCGVSNTTNATTLVGFRGKHTTRAGGNKALRSMEPHVLLVSFPMQGHVNPLLRLGRRLAATGLLVTFTTVRLAAGGGRLRDVPEDGACADVGLGRLRFEYLRDDDDDGDERCQQLAPNDVLSHVTAVGPSALAEFIDGQADAGRPVTFVVNNIFVPWALDVAAGMGIPCAMLWIQPCSVLSIYYHFYESPEAFPTAADPDVPVELPGLPVMAMVELPFMVRPEYAQCLWGDTLRAQVGAIKRTVSWVLVNSFYELERSAVDALRAHTTVKLAPIGPLLEHGHDNGGGDDGAPAPALGAEDNDRCVAWLDAQPPRSVVYVAFGSLVNIGRDETAAVAEGLVATGRPFLWVVRDDSRDLVPEAVLAACRGDKAGKITAWCPQGRVLAHGAVGCFVTHCGWNSIMEALAAGVPVVGYPWWSDQFANAKFLVEDYKVGVRLPAPVTGGELRACVDRVMSGPEAAVIRKRAMHWKREAAAAVADGGSSDRSLQDFVDHVRRSKGPEELARLAQDIQIMNGPVNPVLV

SEQ ID NO: 9 - protein, 477 aa, synthetic construct
MGSLTNNDNLHIFLVCFIGQGVVNPMLRLGKAFASKGLLVTLSAPEIVGTEIRKANNLNDDQPIKVGSGMIRFEFFDDGWESVNGSKPFDVWVYINHLDQTGRQKLPIMLKKHEETGTPVSCLILNPLVPWVADVADSLQIPCATLWVQSCASFSAYYHYHHGLVPFPTESEPEIDVQLPGMPLLKYDEVPDYLHPRTPYPFFGTNILGQFKNLSKNFCILMDTFYELEHEIIDNMCKLCPIKPIGPLFKIPKDPSSNGITGNFMKVDDCKEWLDSRPTSTVVYVSVGSVVYLKQEQVTEMAYGILNSEVSFLWVLRPPSKRIGTEPHVLPEEFWEKAGDRGKVVQWSPQEQVLAHPATVGFLTHCGWNSTQEAISSGVPVITFPQFGDQVTNAKFLVEEFKVGVRLGRGELENRIITRDEVERALREITSGPKAEEVKENALKWKKKAEETVAKGGYSERNLVGFIEEVARKTGTK

SEQ ID NO: 10 - protein, 499 aa, synthetic construct
MGSESLVHVLLVSFPGQGHVNPLLRLGKRLASKGLLVTFTTPETTGKQMRKASNVSEEPTAVGDGYIRFEFFEDGWDEDEPRRQDLDQYLPQLEKVGKIVIPEMIKRNAEQNRPVSCLINNPFIPWVSVVAESLGLPSAMLWVQSCACFAAYYHYHHGLVSFPSETDPEIDVQLPSMPLLKYDEVPSFLHPTTPYPFLRRAILGQYKSLDKPFCILMESFQELEPEIIEYMSKLCPIKPVGPLFKNPKAPNSAVRGDLMPQADDCIDWLNTKPPSSVVYISFGSVVYLKQEQVDEIAHALLNSGITFLWVMKPPHKDSGYELLTLPEGFLEKVGNNGQVVQWSPQEKVLAHPAVACFVTHCGWNSTMESLSSGMPVVAFPQWGDQVTDAVYLTEVFKTGVRMCRGEAEDRIIARDEVEKCLKEAMVGPKAMEMKQNALKWKAAAEAAIAEGGSSDRNLQAFVDEVKRRSMEIAAGKSTKGVGGDLTKKSTENGKVEVEA

SEQ ID NO: 11 - protein, 502 aa, synthetic construct
MDQNSFPLPPHVMLVSFPGQGHVNPLLRLGKLLASKGLLVTFVTTESWGKKMRTANNIQDRVLKPIGKGYIRFDFFDDGLPEDDDASRTDFTILRPQLELVGQREIKSLVRRYEEVTKQPVTCLINNPFVSWVCDVAEDLQIPCAVLWVQSCACLASYYYYHHKLVDFPTKTDPKIDVQIPGMPLLKHDEIPSFIHPSSPYSALREVIIDQIKRLHKPFAVLIDTIHSLEQDIIDHMSDVRLPGVIRPIGPLYKMAKTLASDEINGDMSESTDHCMEWLDSQPVSSVVYISFGTVAYLKQEQINEIAYGVLNAGVSFLWVIRTQELGVNKERHVLPEEVKGKGKIVEWCSQEKVLAHPSIVCFVTHCGWNSTMEAVSSGVPTVCCPQWGDQVTDAVYMIDVLKTGVRLCRGETEERVVPREEVAERLREITKGEKATELKKNALKWKEEAEAAVARGGSSDRNIEEFVEKLGAKRVGKQNGGLEKQNGGLAKQNGSLNHVLS

SEQ ID NO: 12 - protein, 482 aa, synthetic construct
MSAREETAPAAAAAAPPHVVLVCFPSQGNLNPTLRLAKRLAAKGLLVTCCTTSSIGACLAAASSSGVVSTGGDGVRVGSGRIRFEFLDDHGNEKDDLMRYFETSAPAAFVELLGRQAEAGRPVTYVVGNPFLPWVVDVAAEAGVPAAVLWVQSCAVLSLYYHYARGLVEFPPEDDIDARVALPGLPPLSVADVPSFLLPSNPYKMIADAIQGQFRNVDKAAWVFVNSFTELERDVLAALPGVTPRPPQLIPVGPLIELEEDGAAVRGDLIKAADDDCVGWLDAQPPRSVVYASVGSVVVLSAEEVAEMAHGLASTGRPFLWVVRPDTRPLLPEGFLDTVAGCGMVVPWSPQERVLEHAATACFLTHCGWNSTLETVAAGVPVVAFPQWGDQCTDAKFLVDELRMGVRLRAPLRREAVREAVDAAVAGPEADAMLSRARSWSAVARGAVAPGGSSDRHVQAFVDEVVQRACGRQPDEVTMMSG

SEQ ID NO: 13 - protein, 504 aa, synthetic construct
MGSETQIIHNNELDQLVHVFMISFPGQGHINPLLRLGKRIASKGFLVTFTTTENLGQGIRGSNDAVSDQPVPIGDGFIRFEFFDDEWPDGDPKKFDMDQYLPQLEDVGRRWVPERLAVLAQEGRPVSCLINNPFIPWVSDVAEELGLPSAMLWPQSCACFLAYYYFHHNMIPFPTEDALEIDVEVPTLPLLKWDEIPTFLHPTTPYAFLKRAILAQYKNLSKPFCILMDTFYELERSTVDHTIELLAPTPIRTIGPLFKKPIPGGARVRADPLRPNQDCLKWLDGKPDGSVVYISFGTIVFPPQKQIDEIAAGIEAAGITFLWVMKPPAKEAKMSPHTLPDGFLDRIGDNGKVVQFAPQEQVLAHPAVSCFVTHCGWNSTMESLSYGVPMVAFPSWGDQVTDAKFICDVFKTGIQLTRGEHEKRLITKEEVEKCLRDATSGPKAVEMKKIALKWKDLADEAIADGGSSDKNFDFFIDQVRKRGEIVVANAKKVANGLPTKENGH

SEQ ID NO: 14 - protein, 504 aa, synthetic construct
MGSETHHSNDPQLTHIFMISFPGQGHINPLLRLGKRVASKGLLVTFATTENFGQYIRISNDAISDQPVPVGDGFIRLEFFDDEWPDGDPRKHDMDQYLPQLEKVGRKWVTQRLAALAHEYRPVSCLVNNPFLPWVSDLAEELGLCSAMLWPQSCACFLAYYYFHNNLVPFPSQDALEIDVEIPTLPLLKWDEIPTFLHPTTPYAFLKRAILAQYNNLTKPFCVLMDTFYELEKPTVDHTIELLAPLPIKPVGPLFKKKVTGGSDVRADPIRPDQDCLSWLDGQPDGSVIYISFGTVVFLPQKQVDEIAAALEAADLSFLWVMKPPLKESGWTPHCLPDGFLERVGQNGKVVQFAPQEQVLAHPALACFMTHCGWNSTMESLTSGVPVIAFPSWGDQVTDAKFLCDVYKTGIQLTRGEHEKKIIPRDEVEKCLREATSGPKAEEMKENALKWKAHAEETIADGGSSDQNIDFFVEGVRKRSEVVLAKATNGVDANGYSLHIEANG

SEQ ID NO: 15 - protein, 506 aa, synthetic construct
MGSESSLVHVFLVSFPGQGHVNPLLRLGKRLASKGLLVTFTTPESIGKQMRKASNISDQPAPVGDGFIRFEFFEDGWDEDEPRRQDLDQYLPQLEKVGKVLIPQMIQKNAEQGRPVSCLINNPFIPWVSDVAETLGLPSAMLWVQSCACFLAYYHYYHGLVPFPSENAMEIDVQLPSMPLLKHDEVPSFLYPTTPYPFLRRAILGQYKNLEKPFCILMDTFQELEHEIIEYTSKICPIKTVGPLFKNPKAPNTTVKGDFMKADDCIGWLDSKPASSVVYVSFGSVVYLKQDQWDEIAYGLLNSGVNFLWVMKPPHKDSGYTVLTLPEGFLEKAGDRGKVVQWSPQEQVLAHPATACFVTHCGWNSSMEALTSGMPVVAFPQWGDQVTDAKYLVDEFKVGVRMCRGEAEDKLITRDVVEQCLREATQGPKAAEMKKNALKWKAAAEASFVEGGSSDRNLQAFVDEVKRRSIEITASKPAVKAAPNGVVAAAESVVETKANGKVELAA

SEQ ID NO: 16 - protein, 793 aa, synthetic construct
MIEALRQQLLDDPRSWYAFLRHLVASQRDSWLYTDLQRACADFREQLPEGYAEGIGPLEDFVAHTQEVIFRDPWMVFAWRPRPGRWIYVRIHREQLALEELSTDAYLQAKEGIVGLGAEGEAVLTVDFRDFRPVSRRLRDESTIGDGLTHLNRRLAGRIFSDLAAGRSQILEFLSLHRLDGQNLMLSNGNTDFDSLRQTVQYLGTLPRETPWAEIREDMRRRGFAPGWGNTAGRVRETMRLLMDLLDSPSPAALESFLDRIPMISRILIVSIHGWFAQDKVLGRPDTGGQVVYILDQARALEREMRNRLRQQGVDVEPRILIATRLIPESDGTTCDQRLEPVVGAENVQILRVPFRYPDGRIHPHWISRFKIWPWLERYAQDLEREVLAELGSRPDLIIGNYSDGNLVATLLSERLGVTQCNIAHALEKSKYLYSDLHWRDHEQDHHFACQFTADLIAMNAADIIVTSTYQEIAGNDREIGQYEGHQDYTLPGLYRVENGIDVFDSKFNIVSPGADPRFYFSYARTEERPSFLEPEIESLLFGREPGADRRGVLEDRQKPLLLSMARMDRIKNLSGLAELYGRSSRLRGLANLVIIGGHVDVGNSRDAEEREEIRRMHEIMDHYQLDGQLRWVGALLDKTVAGELYRVVADGRGVFVQPALFEAFGLTVIEAMSSGLPVFATRFGGPLEIIEDGVSGFHIDPNDHEATAERLADFLEAARERPKYWLEISDAALARVAERYTWERYAERLMTIARIFGFWRFVLDRESQVMERYLQMFRHLQWRPLAHAVPME

SEQ ID NO: 17 - protein, 808 aa, synthetic construct
MAAKLARLHSLRERLGATFSSHPNELIALFSRYVNQGKGMLQRHQLLAEFDALIEADKEKYAPFEDILRAAQEAIVLPPWVALAIRPRPGVWDYIRVNVSELAVEELSVSEYLAFKEQLVDGHTNSNFVLELDFEPFNASFPRPSMSKSIGNGVQFLNRHLSSKLFQDKESLYPLLNFLKAHNHKGTTMMLNDRIQSLRGLQSSLRKAEEYLMGIPQDTPYSEFNHRFQELGLEKGWGDCAKRVLDTIHLLLDLLEAPDPANLEKFLGTIPMMFNVVILSPHGYFAQSNVLGYPDTGGQVVYILDQVRALENEMLLRIKQQGLDITPKILIVTRLLPDAVGTTCGQRVEKVIGTEHTDILRVPFRSENGILRKWISRFDVWPFLETYTEDVANEIMREMQAKPDLIIGNYSDGNLVATLLAHKLGVTQCTIAHALEKTKYPNSDIYLDKFDSQYHFSCQFTADLIAMNHTDFIITSTFQEIAGSKDTVGQYESHIAFTLPGLYRVVHGIDVFDPKFNIVSPGADMSVYFPYTEADKRLTAFHPEIEELLYSEVENDEHKFVLKDKNKPIIFSMARLDRVKNMTGLVEMYGKNAHLRDLANLVIVCGDHGNQSKDREEQAEFKKMYGLIDQYKLKGHIRWISAQMNRVRNGELYRYICDTKGVFVQPAFYEAFGLTVIEAMTCGLPTIATCHGGPAEIIVDGVSGLHIDPYHSDKAADILVNFFEKCKQDSTYWDNISQGGLQRIYEKYTWKLYSERLMTLTGVYGFWKYVSNLERRETRRYIEMFYALKYRSLASAVPLAVDGESTSK

SEQ ID NO: 18 - protein, 794 aa, synthetic construct
MTTIDTLATCTQQNRDAVYTLLRRYFTANRTLLLQSDLREGLLQTEQDCGQSDMLRAFVFRLQEGIFSSPWAYLALRPEIAKWEFMRIHQEHLIPEKLTISEFLKFKETVVKGEATESVLEVDFGPFNRGFPRLKESRSIGQGVIFLNRKLSSEMFSRIEAGHTSLLHFLGVHAIEGQQLMFSNNSHDIHAVRNQLRQALEMLETLDGTTPWIELAPKMNQLGFAPGWGHNANRVAETMNMLMDILEAPSPSALEEFLACIPMISRLLILSPHGYFGQDNVLGLPDTGGQVVYILDQVRALEKEMHDRLQLQGVQVEPKILIVTRLIPDAGDTTCNQRLEKVSGCTNTWILRVPFRKHNGEIIPHWISRFEIWPHLEIFAGDVEREALAELGGHPDLIIGNYSDGNLVATLLSRRLGVTQCNIAHALEKTKYLHSDIYWQENEDKYHFSCQYTADLLAMNSADFIVTSTYQEIAGTREAEGQYESYQAFSMPDLYRVIHGIDLFDPKFNIVSPGANADIYFPYSDPNRRLHSLIPEIESLIFDDATNLPARGYLQDPDKPLIFTMARLDRIKNITGLVELYAASPRLRSLANLVIVGGKIDPQHSSDHEEQEQIHRMHQLMDEHELDQQVRWLGMRLDKNLAGELYRYIADKRGIFVQPALFEAFGLTIIEAMASGLPTFATRYGGPLEIIQNNRSGFHIDPNQGAATADLIADFFEKNLENPQEWERISQGALDRVASRYTWKLYAERMMTLSRIYGFWKFVSGLEREETDRYLNMFYHLQFRPLANRLAHEI

SEQ ID NO: 19 - protein, 805 aa, synthetic construct
MATDRLTRVHSLRERLDETLTANRNEILALLSRIEAKGKGILQHHQVIAEFEEIPEENRQKLTDGAFGEVLRSTQEAIVLPPWVALAVRPRPGVWEYLRVNVHALVVEELQPAEYLHFKEELVDGSSNGNFVLELDFEPFNAAFPRPTLNKSIGNGVQFLNRHLSAKLFHDKESLHPLLEFLRLHSVKGKTLMLNDRIQNPDALQHVLRKAEEYLGTVPPETPYSEFEHKFQEIGLERGWGDNAERVLESIQLLLDLLEAPDPCTLETFLGRIPMVFNVVILSPHGYFAQDNVLGYPDTGGQVVYILDQVRALENEMLHRIKQQGLDIVPRILIITRLLPDAVGTTCGQRLEKVFGTEHSHILRVPFRTEKGIVRKWISRFEVWPYLETYTEDVAHELAKELQGKPDLIVGNYSDGNIVASLLAHKLGVTQCTIAHALEKTKYPESDIYWKKLEERYHFSCQFTADLFAMNHTDFIITSTFQEIAGSKDTVGQYESHTAFTLPGLYRVVHGIDVFDPKFNIVSPGADQTIYFPHTETSRRLTSFHPEIEELLYSSVENEEHICVLKDRSKPIIFTMARLDRVKNITGLVEWYGKNAKLRELVNLVVVAGDRRKESKDLEEKAEMKKMYGLIETYKLNGQFRWISSQMNRVRNGELYRVICDTRGAFVQPAVYEAFGLTVVEAMTCGLPTFATCNGGPAEIIVHGKSGFHIDPYHGDRAADLLVDFFEKCKLDPTHWDKISKAGLQRIEEKYTWQIYSQRLLTLTGVYGFWKHVSNLDRRESRRYLEMFYALKYRKLAESVPLAAE

SEQ ID NO: 20 - protein, 806 aa, synthetic construct
MSELMQAILDSEEKHDLRGFISELRQQDKNYLLRNDILNVYAEYCSKCQKPETSYKFSNLSKLIYYTQEIIPEDSNFCFIIRPKIAAQEVYRLTADLDVEPMTVQELLDLRDRLVNKFHPYEGDILELDFGPFYDYTPTIRDPKNIGKGVQYLNRYLSSKLFQDSQQWLESLFNFLRLHNYNGIQLLINHQIQSQQQLSQQVKNALNFVSDRPNDEPYEQFRLQLQTMGFEPGWGNTASRVRDTLNILDELIDSPDPQTLEAFISRIPMIFRIVLVSAHGWFGQEGVLGRPDTGGQVVYVLDQAKNLEKQLQEDAILAGLEVLNVQPKVIILTRLIPNSDGTLCNQRLEKVYGTENAWILRVPLREFNPKMTQNWISRFEFWPYLETFAIDSERELLAEFQGRPDLIVGNYTDGNLVAFLLTRRMKVTQCNIAHALEKSKYLFSNLYWQDLEEKYHFSLQFTADLIAMNAANFVISSTYQEIVGTPDSIGQYESYKCFTMPELYHVVNGIELFSPKFNVVPPGVNENSYFPYTQTQNRIESDRDRLEEMLFTLEDSSQIFGKLDDPNKRPIFSMARLDRIKNLTGLAECFGQSQELQERCNLILVAGKLRIEESEDNEEKDEIVKLYRIIDEYNLHGKIRWLGVRLSKNDSGEIYRVICDRQGIFVQPALFEAFGLTILESMISGLPTFATQFGGPLEIIQDKINGFYINPTHLEETATKILDFVTKCEQNPNYWNIISEKAIDRVYSTYTWKIHTTKLLTLARIYGFWNFTSKEKREDLLRYLESLFYLIYKPRAQQLLEQHKYR

SEQ ID NO: 21 - protein, 816 aa, synthetic construct
MGEGAGDRVLSRLHSVRERIGDSLSAHPNELVAVFTRLKNLGKGMLQPHQIIAEYNNAIPEAEREKLKDGAFEDVLRAAQEAIVIPPWVALAIRPRPGVWEYVRVNVSELAVEELRVPEYLQFKEQLVEEGPNNNFVLELDFEPFNASFPRPSLSKSIGNGVQFLNRHLSSKLFHDKESMYPLLNFLRAHNYKGMTMMLNDRIRSLSALQGALRKAEEHLSTLQADTPYSEFHHRFQELGLEKGWGDCAKRAQETIHLLLDLLEAPDPSTLEKFLGTIPMVFNVVILSPHGYFAQANVLGYPDTGGQVVYILDQVRAMENEMLLRIKQCGLDITPKILIVTRLLPDATGTTCGQRLEKVLGTEHCHILRVPFRTENGIVRKWISRFEVWPYLETYTDDVAHEIAGELQANPDLIIGNYSDGNLVACLLAHKMGVTHCTIAHALEKTKYPNSDLYWKKFEDHYHFSCQFTTDLIAMNHADFIITSTFQEIAGNKDTVGQYESHMAFTMPGLYRVVHGIDVFDPKFNIVSPGADLSIYFPYTESHKRLTSLHPEIEELLYSQTENTEHKFVLNDRNKPIIFSMARLDRVKNLTGLVELYGRNKRLQELVNLVVVCGDHGNPSKDKEEQAEFKKMFDLIEQYNLNGHIRWISAQMNRVRNGELYRYICDTKGAFVQPAFYEAFGLTVVEAMTCGLPTFATAYGGPAEIIVHGVSGYHIDPYQGDKASALLVDFFDKCQAEPSHWSKISQGGLQRIEEKYTWKLYSERLMTLTGVYGFWKYVSNLERRETRRYLEMLYALKYRTMASTVPLAVEGEPSSK

SEQ ID NO: 22 - protein, 808 aa, synthetic construct
MANAERMITRVHSQRERLNETLVSERNEVLALLSRVEAKGKGILQQNQIIAEFEALPEQTRKKLEGGPFFDLLKSTQEAIVLPPWVALAVRPRPGVWEYLRVNLHALVVEELQPAEFLHFKEELVDGVKNGNFTLELDFEPFNASIPRPTLHKYIGNGVDFLNRHLSAKLFHDKESLLPLLKFLRLHSHQGKNLMLSEKIQNLNTLQHTLRKAEEYLAELKSETLYEEFEAKFEEIGLERGWGDNAERVLDMIRLLLDLLEAPDPCTLETFLGRVPMVFNVVILSPHGYFAQDNVLGYPDTGGQVVYILDQVRALEIEMLQRIKQQGLNIKPRILILTRLLPDAVGTTCGERLERVYDSEYCDILRVPFRTEKGIVRKWISRFEVWPYLETYTEDAAVELSKELNGKPDLIIGNYSDGNLVASLLAHKLGVTQCTIAHALEKTKYPDSDIYWKKLDDKYHFSCQFTADIFAMNHTDFIITSTFQEIAGSKETVGQYESHTAFTLPGLYRVVHGIDVFDPKFNIVSPGADMSIYFPYTEEKRRLTKFHSEIEELLYSDVENKEHLCVLKDKKKPILFTMARLDRVKNLSGLVEWYGKNTRLRELANLVVVGGDRRKESKDNEEKAEMKKMYDLIEEYKLNGQFRWISSQMDRVRNGELYRYICDTKGAFVQPALYEAFGLTVVEAMTCGLPTFATCKGGPAEIIVHGKSGFHIDPYHGDQAADTLADFFTKCKEDPSHWDEISKGGLQRIEEKYTWQIYSQRLLTLTGVYGFWKHVSNLDRLEARRYLEMFYALKYRPLAQAVPLAQDD

SEQ ID NO: 23 - protein, 809 aa, synthetic construct
MANPKLTRVLSTRDRVQDTLSAHRNELVALLSRYVDQGKGILQPHNLIDELESVIGDDETKKSLSDGPFGEILKSAMEAIVVPPFVALAVRPRPGVWEYVRVNVFELSVEQLTVSEYLRFKEELVDGPNSDPFCLELDFEPFNANVPRPSRSSSIGNGVQFLNRHLSSVMFRNKDCLEPLLDFLRVHKYKGHPLMLNDRIQSISRLQIQLSKAEDHISKLSQETPFSEFEYALQGMGFEKGWGDTAGRVLEMMHLLSDILQAPDPSSLEKFLGMVPMVFNVVILSPHGYFGQANVLGLPDTGGQVVYILDQVRALETEMLLRIKRQGLDISPSILIVTRLIPDAKGTTCNQRLERVSGTEHTHILRVPFRSEKGILRKWISRFDVWPYLENYAQDAASEIVGELQGVPDFIIGNYSDGNLVASLMAHRMGVTQCTIAHALEKTKYPDSDIYWKDFDNKYHFSCQFTADLIAMNNADFIITSTYQEIAGTKNTVGQYESHGAFTLPGLYRVVHGIDVFDPKFNIVSPGADMTIYFPYSEETRRLTALHGSIEEMLYSPDQTDEHVGTLSDRSKPILFSMARLDKVKNISGLVEMYSKNTKLRELVNLVVIAGNIDVNKSKDREEIVEIEKMHNLMKNYKLDGQFRWITAQTNRARNGELYRYIADTRGAFAQPAFYEAFGLTVVEAMTCGLPTFATCHGGPAEIIEHGLSGFHIDPYHPEQAGNIMADFFERCKEDPNHWKKVSDAGLQRIYERYTWKIYSERLMTLAGVYGFWKYVSKLERRETRRYLEMFYILKFRDLVKTVPSTADD

SEQ ID NO: 24 - protein, 1048 aa, synthetic construct
MAGNDWINSYLEAILDVGPGIDDAKSSLLLRERGRFSPTRYFVEEVITGFDETDLHRSWVRAQATRSPQERNTRLENMCWRIWNLARQKKQLEGEQAQRLAKRRQERERGRREAVADMSEDLSEGEKGDAISDISAHGESIKGRLPRISSVETMESWANQQKGKKLYIVLISLHGLIRGENMELGRDSDTGGQVKYVVELARALGSMPGVYRVDLLTRQVSSPEVDWSYGEPTEMLTPINSEGLMTEMGESSGAYIIRIPFGPRDKYIPKEDLWPYIPEFVDGALNHILHVSKVLGGQIGSGRDVWPVAIHGHYADAGDSAALLSGALNVPMLFTGHSLGRDKLEQLLRQGRLSKDEINSTYKIMRRIEAEELSLDASEIVITSTRQEIDEQWRLYDGFDPILERKLRARIKRNVSCYGRFMPRMVVIPPGMEFHHIVPHEGDMDFETEGSEDGKAPDPHIWTEIMRFFSNPRKPMILALARPDPKKNLTTLVKAFGECRPLRELANLTLIMGNRDNIDEMSSTNASVLLSILKMIDKYDLYGQVAYPKHHKQSEVPDIYRLAAKTKGVFINPAFIEPFGLTLIEAAAHGLPIVATKNGGPVDIHRGSDNGLLVDPHDQHAIADALLKLVADKHLWAKCRANGLKNIHLFSWPEHCKTYLSRIAGCKPRQPCWLRNADDDENSESESPSDSLRDIQDISLNLKFSLDGDKNEDSDNLFDPDDRKNKLENAVLAWSKGVKGTHKTSIDKIDQSSSAGKFPALRRRKQIFVIAVDCDSSTGLFENVRKIFAAVEAEGMEGSIGFHIGHFIQYIRSAFFSDFRGHESTDFDAFICNSGGDLYYSSSHSEDNPFVVDLYYHSHIEYRWGGEGLRKTLVRWAASISDKKGEKEEHIVVEDEKNSADYCYTFKVQKSGGDPSVKELRKSMRIQALRCHVVYCQNGSRINVIPVLSSRSQALRYLYLRWGMDLSKLVVFVGESGDTDYEGLLGGLRKAVILKGVCSVSSSQLLSNRNYPLTDVVPYNSPNVIQTTEECSSSELHASLEKLAVLKG

SEQ ID NO: 25 - protein, 808 aa, synthetic construct
MTCVLLKAVVESDERADLRQFSRILQLGEKRYLLRNDILDAFADYCRDQERPVPPPSESRLSKLVFYTQEIIVDNESLCWIVRPRIAQQEVCRLLVEDLTIVPMTIPELLDLRDRLVNHYHPNEGDVFEIDVQPFYDYSPIIRDAKNIGKGVEFLNRYLSSKLFQDPRQWQQNLFNFLRIHRYNGYQLLINERIRSPQHLSEQVKQALVVLSDRPPTEAYSEFRFELQNLGFEPGWGNTVARVRDTLEILDQLLDSPDHQVLEAFVSRIPMLFRIALISPHGWFGQEGVLGRPDTGGQVVYILDQVKSLEKQMREDLELAGLGVLEAQPKIIVLTRLIPNAEGTLCNQRLEKIYGTNDAWILRVPFREFNPKVTQNWISRFEIWPYLETFAIDAERELRAEFGHVPDLIIGNYSDGNLVAFLLARRLKVTQCNIAHALEKSKYLFSNLYWQDLEDKYHFSLQFTADLIAMNAANFIISSTYQEIVGTPDSIGQYESYQSFTMPDLYHVVNGIELFSPKFNVVPPGVNEQVYFPYYHYTERLEGDRQRLEELLFTLEDPQQIYGYLEAPEKRPLFSMARLDRIKNLTGLAEAFGRSKALQERCNLILVAGKLRTADSSDREEIAEIEKLYQIIHQYNLHGKIRWLGIRLPKADSGEIYRIIADRQGIFVQPALFEAFGLTILEAMISGLPTFGTRFGGPLEIIQDGVNGFYINPTHLEEMAETIVRFLEACDRDPQEWQRISKAGIERVYSTYTWKIHCTRLLSLAKIYGFWNFSSQENREDMMRYMEALFHLLYKPRAQALLAEHLQR

SEQ ID NO: 26 - protein, 327 aa, synthetic construct
MEIVAIDIGGTHARFSIAEVSNGRVLSLGEETTFKTAEHASLQLAWERFGEKLGRPLPRAAAIAWAGPVHGEVLKLTNNPWVLRPATLNEKLDIDTHVLINDFGAVAHAVAHMDSSYLDHICGPDEALPSDGVITILGPGTGLGVAHLLRTEGRYFVIETEGGHIDFAPLDRLEDKILARLRERFRRVSIERIISGPGLGNIYEALAAIEGVPFSLLDDIKLWQMALEGKDNLAEAALDRFCLSLGAIAGDLALAQGRTSVVIGGGVGLRIASHLPESGFRQRFVSKGRFERVMSKIPVKLITYPQPGLLGAQLPMPTNILKLNNIF

SEQ ID NO: 27 - protein, 322 aa, synthetic construct
MEIVAVDIGGTHARFAIAEVGDGHVLSLGEPVTLKTAEHGSLQLAWEAAGEALGRPIPRAAGIAIATPIGGEVLKLTNNPWVIRPALIQSKLGVDSYVLVNDFEAVGHAVAQVGAEYLQHLCGPDDPLPDTGIITIVGPGTGLGVAHLLKTPAGYHVLPCEGGHIDFAPLDVLEDGILKTLRKQYRRVSVERIVSGPGLVNIYESLPPSTSAALRPIDDKELWTAALAGTDSRAAAALDRFCLSLGAVAGDLALAHGAKAVVIAGGLGLRIADHLPRSGFSERFVAKGRFERIMSAMPVKFITHPQPGLFGAAAAFAGAHRS

SEQ ID NO: 28 - protein, 546 aa, synthetic construct
MAIHNRAGQPAQQSDLINVAQLTAQYYVLKPEAGNAEHAVKFGTSGHRGSAARHSFNEPHILAIAQAIAEERAKNGITGPCYVGKDTHALSEPAFISVLEVLAANGVDVIVQENNGFTPTPAVSNAILVHNKKGGPLADGIVITPSHNPPEDGGIKYNPPNGGPADTNVTKVVEDRANALLADGLKGVKRISLDEAMASGHVKEQDLVQPFVEGLADIVDMAAIQKAGLTLGVDPLGGSGIEYWKRIGEYYNLNLTIVNDQVDQTFRFMHLDKDGAIRMDCSSECAMAGLLALRDKFDLAFANDPDYDRHGIVTPAGLMNPNHYLAVAINYLFQHRPQWGKDVAVGKTLVSSAMIDRVVNDLGRKLVEVPVGFKWFVDGLFDGSFGFGGEESAGASFLRFDGTPWSTDKDGIIMCLLAAEITAVTGKNPQEHYNELAKRFGAPSYNRLQAAATSAQKAALSKLSPEMVSASTLAGDPITARLTAAPGNGASIGGLKVMTDNGWFAARPSGTEDAYKIYCESFLGEEHRKQIEKEAVEIVSEVLKNA

SEQ ID NO: 29 - protein, 554 aa, synthetic construct
MAKHPRAGQQPTSCDLINVAKLTSQYYVLQPALHESAHAVKFGTSGHRGSSTKHTFNEAHILAIAQAIAEIRHAKGITGPCYVGKDTHALSEPAFISVLEVLAANQVTVIIQPENGFTPTPAISHAILTHNKSNPIHLADGIVITPSHNPPEDGGIKYNPPNGGPADTDFTGLIEKRANQLLDERSSENAVLAGVKRISLSQAISSQFIIQQDMMMPYVEDLVNVVDLSVIQQAGLKLGVDPLGGSGIAYWQQIAEYYKLNLSLVNDQVDQTFRFMPLDHDGVIRMDCSSVDAMGGLLRLKDKFDLAFANDPDYDRHGIVTPAGLMNPNHYLSVAINYLYQNRPGWPVDLGVGKTLVSSAMIDRVVSSLNRKLVEVPVGFKWFVDGLYTGKLGFGGEESAGASFLRFDAKPWSTDKDGIIMCLLSAEITARTGKNPQVHYDELSEKFGAPIYNRIQANATHEQKAILSKLSPDQVQAKTLAGDLITARITHAPSNNASIGGLKVVTDNGWFAARPSGTEEAYKIYCESFISHEHLDLISKEAVEIVDAAFASNK

SEQ ID NO: 30 - protein, 548 aa, synthetic construct
MAMHPRAGQKAQQEDLHNIPALVANYFLLQPDAANAEHKVQFGTSGHRGTADKHTFNENHILAIAQAVAEVRSEQGTTGPLFVGKDTHALSEPAFSSVIEVLIANGVKVIVQQDNGYTPTPGISHAILTYNIKHDEKADGIVITPSHNPPQDGGIKYNPTHGGPAEAELTQAIEDRANELIAEGLQGVKRLPLAEAKASDLFVEMDLVKPYIDDLVNVIDMEAIQKANLKIGVDPLGGSGIDYWRQIGQAYNLDLTLVSEAIDPSFQFMSLDKDGVVRMDCSSPYAMAGLLALKDEYDLAFGNDPDYDRHGIVTPKGLMNPNHFLAVCIDYLYRHRDAWGKDVAVGKTLVSSALIDRVVADLGRALCEVPVGFKWFVDGLYTGKFGFGGEESAGASFLRKDGTPWSTDKDGILLCLLAAEITAVTGKNPQDYYEELAAKHGESKYNRIQAVANGPQKDVLKKLSPEMVAAETLAGDAITARLTHAPGNGAAIGGLKVTTANGWFAARPSGTEDIYKIYCESFKGEEHLKQIEAEAQEIVNQVFAAAGL

SEQ ID NO: 31 - protein, 302 aa, synthetic construct
MAAINTKVKKAVIPVAGLGTRMLPATKAIPKEMLPLVDKPLIQYVVNECIAAGITEIVLVTHSSKNSIENHFDTSFELEAMLEKRVKRQLLDEVQSICPPHVTIMQVRQGLAKGLGHAVLCAHPVVGDEPVAVILPDVILDEYESDLSQDNLAEMIRRFDETGHSQIMVEPVADVTAYGVVDCKGVELAPGESVPMVGVVEKPKADVAPSNLAIVGRYVLSADIWPLLAKTPPGAGDEIQLTDAIDMLIEKETVEAYHMKGKSHDCGNKLGYMQAFVEYGIRHNTLGTEFKAWLEEEMGIKK

SEQ ID NO: 32 - protein, 488 aa, synthetic construct
MFAEDLKRTEKMTVDDVFEQSAQKMREQGMSEIAISQFRHAYHVWASEKESAWIREDTVEPLHGVRSFHDVYKTIDHDKAVHAFAKTAFLKLNGGLGTSMGLQCAKSLLPVRRHKARQMRFLDIILGQVLTARTRLNVPLPVTFMNSFRTSDDTMKALRHQRKFKQTDIPLEIIQHQEPKIDAATGAPASWPANPDLEWCPPGHGDLFSTLRESGLLDTLLEHGFEYLFISNSDNLGARPSRTLAQYFEDTGAPFMVEVANRTYADRKGGHIVRDTATGRLILREMSQVHPDDKDAAQDIAKHPYFNTNNIWVRIDVLRDMLAEHDGVLPLPVIINNKTVDPIDPQSPAVVQLETAMGAAIGLFEGAICVQVDRMRFLPVKTTNDLFIMRSDRFHLTDSYEMEDGNYIFPNVDLDPRYYKNIEDFNERFPYNVPSLAAANSVSIKGDWTFGRDVIMFADARLEDRNEPSYVPNGEYVGPMGIEPGDWV

SEQ ID NO: 33 - protein, 299 aa, synthetic construct
MTASAEHQTFTTAIVPAAGLGTRFLPTTKSVPKELLPVVDTPAIELVADEARQAGAERLVIVTSPAKQSIAAYFRPAPELERSLEEKGKTGQLAKIRRAPELLEVEVAIQEQALGLGHAVACAEPNLGPEDDVVAVLLPDDLVLPHGILERMAKVRAEHGGSVLCAFDIPKEEISAYGVFDVSDTDDADVKRVHGMVEKPPAEQAPSTFAAAGRYLLDRAIFDALRRIEPGAGGELQLTDAVALLIQEGHPVHVVVHRGDRHDLGNPGGFLRAAVDFALQDPDYGPELRAWLTDRIARP

SEQ ID NO: 34 - protein, 143 aa, synthetic construct
MAIERTFSIIKPNAVAKNVIGNIFARFEAAGFKIVGTKMLHLTVEQARGFYAEHDGKPFFDGLVEFMTSGPIVVSVLEGENAVQRHRDLLGATNPANALAGTLRADYADSLTENGTHGSDSVESAAREIAYFFGEGEVCPRTR

SEQ ID NO: 35 - protein, 136 aa, synthetic construct
MTERTLILIKPDGVTNGHVGEIIARIERKGLKLAALDLRVADRETAEKHYEEHADKPFFGELVEFITSAPLIAGIVEGERAIDAWRQLAGGTDPVAKATPGTIRGDFALTVGENVVHGSDSPESAEREISIWFPNL

SEQ ID NO: 36 - protein, 136 aa, synthetic construct
MTERTLILIKPDGVANGHVGEIIARIERKGLKLVELDLRTADRETAEKHYEEHSDKPFFGELVEFITSAPLVAGIVEGERAIDAWRQLAGGTDPVSKATPGTIRGDFALTVGENVVHGSDSPESAEREIAIWFPNK

SEQ ID NO: 37 - protein, 300 aa, synthetic construct
MALDEAPAEARPGSRAVELEIDGRSRIFDIDDPDLPKWIDEEAFRSDDYPYKKKLDREEYEETLTKLQIELVKVQFWMQATGKRVMAVFEGRDAAGKGGAIHATTANMNPRSARVVALTKPTETERGQWYFQRYVATFPTAGEFVLFDRSWYNRAGVEPVMGFCTPDQYEQFLKEAPRFEEMIANEGIHLFKFWINIGREMQLKRFHDRRHDPLKIWKLSPMDIAALSKWDDYTGKRDRMLKETHTEHGPWAVIRGNDKRRSRINVIRHMLTKLDYDGKDEAAIGEVDEKILGSGPGFLR

SEQ ID NO: 38 - protein, 293 aa, synthetic construct
MVEKAESRAVELDIDGKKRVFDIDDPKLPDWIDKEAFQSDDFPYDKKLDEDEYEEALEALQVELVKVQFWQQKTGARIMAIFEGRDAAGKGGAIHATMSNMNPRSARIVALTKPTETEQGQWYFQRYVATFPTSGEFVLFDRSWYNRAGVEPVMGFCTPEQYEKFLKATPRIEKMIASEGIHFFKFWLNIGREMQLKRFHDRRHDPLKVWKLSPMDIAALNKWDDYTEKRDRMLKATHTDRAPWTVIRANDKRRARINLIRHILTTLDYEGKDEKAIGKIDDKIVGSGPGFVK